(19) United States
(12) Patent Application Publication
(10) Pub. No.: US 2022/0241367 A1
(43) Pub. Date: Aug. 4, 2022

(54) Treatment of Abdominal Pain Associated with Diarrhea-Predominant Irritable Bowel Syndrome

(71) Applicant: Ironwood Pharmaceutical, Inc., Boston, MA (US)

(72) Inventor: Wilmin Bartolini, Amesbury, MA (US)

(21) Appl. No.: 17/617,548

(22) PCT Filed: Jun. 9, 2020
(86) PCT No.: PCT/US20/36767

§ 371 Date: Dec. 8, 2021

Related U.S. Application Data
(60) Provisional application No. 62/859,443, filed on Jun. 10, 2019.

(51) Int. Cl. A61K 38/10; A61K 9/00; A61K 9/20; A61P 29/00

(52) U.S. Cl. CPC A61K 38/10; A61K 9/0053; A61P 29/00; A61K 9/2027; A61K 9/2013; A61K 9/2009

(57) ABSTRACT

The invention provides methods for treating a patient with a disorder, such as a GI disorder or symptoms associated with a GI or non-GI disorder, by administering a therapeutically effective amount of a delayed release pharmaceutical composition comprising linaclotide.

### CROSS REFERENCE TO RELATED APPLICATIONS

This application claims priority under 35 U.S.C. § 119(e) to U.S. Provisional Patent Application 62/859,443, filed Jun. 10, 2019. The entire contents of the aforementioned application are hereby incorporated by reference in its entirety, including drawings.

### FIELD OF THE INVENTION

The invention relates to the use of pharmaceutical compositions comprising linaclotide to treat a variety of indications, including gastrointestinal (GI) disorders, such as irritable bowel syndrome with diarrhea (IBS-d) and symptoms associated with GI or non-GI disorders, such as abdominal pain.

### BACKGROUND OF THE INVENTION

Irritable bowel syndrome (IBS) is a chronic functional gastrointestinal (GI) disorder characterized by recurrent abdominal pain associated with defecation and/or a change in stool frequency or form. In addition to the characteristic abdominal pain, IBS is often associated with abdominal distension and bloating. In moderate to severe cases of IBS, an overall deterioration in quality of life (QOL) is often present. IBS is one of the most frequently seen disorders in the United States; data suggest the prevalence of IBS is 11-14% of the adult population. IBS is subtyped based on predominant stool form as IBS with diarrhea (IBS-d), IBS with constipation (IBS-c), or IBS mixed (IBS-M; mixed constipation and diarrhea), according to the Rome diagnostic criteria. IBS patients who rarely or never have abnormal stools or do not fit into 1 of the 3 main IBS subtypes are subtyped as unclassified IBS (IBS-U). Rome IV Diagnostic Criteria for irritable bowel syndrome (IBS) includes Rome IV criteria for IBS: reports recurrent abdominal pain, on average at least 1 day/week during the 3 months before the diagnosis, with the onset at least 6 months before the diagnosis, associated with 2 or more of the following features:

a. Related to defecation

b. Associated with a change in frequency of stool

c. Associated with a change in form (appearance) of stool Patients with IBS-d may also report symptoms that include (i) diarrhea, and (ii) abdominal pain or discomfort.

Rome IV criteria for IBS-d, based on stool form on days with at least 1 abnormal bowel movement (BM): >25% of BMs with Bristol stool form scale (BSFS) score of 6 or 7 and <25% of BMs with BSFS score of 1 or 2.

U.S. Pat. Nos. 7,304,036 and 7,371,727, herein incorporated by reference, disclose peptides that act as agonists of the guanylate cyclase C (GC-C) receptor for the treatment of gastrointestinal (GI) disorders. One particular peptide disclosed is linaclotide, which consists of the following amino acid sequence: Cys Cys Glu Tyr Cys Cys Asn Pro Ala Cys Thr Gly Cys Tyr. Linaclotide has the chemical structure of:

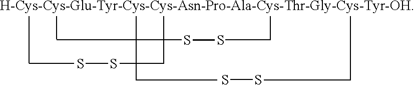

Linaclotide is orally administered and has been approved in the U.S. by the FDA for the treatment of irritable bowel syndrome with constipation (IBS-c) and chronic idiopathic constipation (CIC). As approved by the FDA, linaclotide is administered in an oral, solid, immediate-release capsule formulation manufactured by filling drug-layered beads into gelatin capsules. Due to the high expression of GC-C receptors throughout GI tract, linaclotide from an immediate release formulation activates the GC-C receptor starting from the upper GI tract, resulting in significant amount of intestinal fluid being brought to the lower GI tract. To reduce or mitigate this effect, compositions are needed which have targeted release of linaclotide in the distal or lower segment of the gastrointestinal tract. Targeting the lower GI for linaclotide release may help avoid excess fluid secretion but at the same time maintain or improve linaclotide efficacy for treating abdominal pain and discomfort of GI disorders.

Delayed release (“DR”) compositions of linaclotide that target the lower GI may improve linaclotide's efficacy towards relieving pain associated with various GI disorders by allowing for delivery of a higher dose of linaclotide to the colon. Such DR compositions of linaclotide would have the potential to release linaclotide predominantly (or fully) in the lower GI. As a result, for example, the DR formulation or composition may have an increased capacity to treat lower GI associated disorders. Surprisingly, orally administered linaclotide has also been demonstrated to reduce visceral pain in non-GI tissues, providing further evidence that the mechanism of visceral pain relief via linaclotide is not mediated solely by promoting secretion. This result suggests that a cGMP modulator whose distribution is limited to the GI can relieve pain and may be used as a therapy to relieve pain in other parts of the body. However, in order for linaclotide to be a useful therapy for the treatment of visceral pain in non-GI tissues (e.g., ulcerative colitis, diverticulitis, IBS, overactive bladder syndrome, bladder hypersensitivity or colitis induced bladder afferent hyperactivity, etc.) for non-constipated patients, such as IBS-d patients, it would be necessary to reduce or eliminate the secretion-promoting effects of linaclotide. As such, in one aspect, there is a need to develop a means of at least partially, or completely separating the effect of linaclotide in promoting secretion from that of relieving visceral pain.

Currently, there are very few FDA approved therapies for the treatment of IBS-d. Further, there are no FDA approved therapies specifically for treating abdominal pain or discomfort associated with IBS-d. Therefore, there remains an unmet medical need for additional, well-tolerated, and effective therapies to treat abdominal pain and discomfort associated with IBS-d.

### SUMMARY OF THE INVENTION

In general, the invention relates to a method of treating disorders, such as gastrointestinal (GI) disorders (e.g., IBS-d) or symptoms associated with GI or non-GI disorders (e.g., abdominal pain or discomfort).

Another aspect of the invention is a method of reducing intestinal fluid secretion-promoting effects of linaclotide, comprising orally administering to a subject a delayed-release pharmaceutical tablet composition comprising linaclotide, wherein the tablet further comprises an enteric coating comprising a pH-sensitive polymer that releases linaclotide in a lower GI of the subject.

Yet another aspect of the invention is a method of treating visceral or abdominal pain in a non-constipated subject, comprising orally administering a pharmaceutical tablet composition comprising linaclotide, wherein the tablet further comprises an enteric coating and a pH sensitive polymer that releases linalcotide in the ileum, terminal ileum, or colon.

Another aspect of the invention is a method of treating abdominal pain associated with (IBS) or irritable bowel syndrome with (IBS-d) comprising orally administering to a patient in need thereof, a pharmaceutical tablet composition comprising a therapeutically effective amount of linaclotide.

Still another aspect of the invention is a method of treating abdominal pain comprising orally administering to a patient in need thereof, a delayed release pharmaceutical tablet composition comprising a therapeutically effective amount of linaclotide.

### BRIEF DESCRIPTION OF THE FIGURES

FIG. 1 is a plot of change in SBM frequency: DR2 vs Placebo and linaclotide 290 μg described in Example 10.

FIG. 2 is a plot of change in stool consistency: DR2 vs Placebo and linaclotide 290 μg as described in Example 10.

FIG. 3 is a table of results of change from baseline in bowel movement frequency rate (SBM) on patient outcomes from Example 10.

FIG. 4 is a plot of change from baseline (CFB) in bowel movement frequency for patients administered delayed release compositions of linaclotide.

### DETAILED DESCRIPTION OF THE INVENTION

A. Methods of Treatment

In one aspect, described generally herein are methods of treatment comprising orally administering a composition comprising linaclotide, which are used to treat any number of diseases, disorders or symptoms involving pain (e.g., visceral pain, abdominal pain). For example, the methods of treatment comprising orally administering a delayed release pharmaceutical tablet composition comprising linaclotide, as described herein, are used to treat irritable bowel syndrome with diarrhea (IBS-d) in a patient in need thereof. The patient may be diagnosed with IBS-d according to the Rome Criteria (e.g. Rome II). In another embodiment, the methods of treatment comprising orally administering a pharmaceutical tablet composition comprising linaclotide, as described herein, are used to treat abdominal pain in a patient in need thereof.

Another aspect of the invention is a method of reducing intestinal fluid secretion-promoting effects of linaclotide, comprising orally administering to a subject a pharmaceutical tablet composition comprising linaclotide, wherein the tablet further comprises an enteric coating comprising a pH-sensitive polymer that releases linaclotide in a lower GI of the subject.

Yet another aspect of the invention is a method of treating visceral or abdominal pain in a non-constipated subject, comprising orally administering a pharmaceutical tablet composition comprising linaclotide.

Another aspect of the invention is a method of treating irritable bowel syndrome with diarrhea (IBS-d) comprising orally administering to a patient in need thereof, a pharmaceutical tablet composition comprising a therapeutically effective amount of linaclotide.

Still another aspect of the invention is a method of treating abdominal pain comprising orally administering to a patient in need thereof, a delayed release pharmaceutical tablet composition comprising a therapeutically effective amount of linaclotide.

In some embodiments, the delayed-release pharmaceutical tablet composition comprises a therapeutically effective amount of linaclotide to reduce, prevent or relieve pain or diarrhea in the subject. In some embodiments, the delayed-release pharmaceutical composition comprises a therapeutically effective amount of linaclotide to reduce, prevent or relieve pain in the subject, but does not affect bowel habit. In some embodiments, the delayed-release pharmaceutical composition provides less than an amount of linaclotide effective to substantially affect bowel habit. In some embodiments, the bowel habit is selected from complete spontaneous bowel movement rate, spontaneous bowel movement rate, or stool consistency.

In some embodiments, the subject is diagnosed with irritable bowel syndrome with diarrhea (IBS-d).

In some embodiments, the pharmaceutical tablet composition is administered once daily. In some embodiments, the pharmaceutical tablet composition is administered once daily in the morning. In some embodiments, the pharmaceutical tablet composition is administered once daily in the morning at least 30 minutes after breakfast. In some embodiments, the pharmaceutical tablet composition is administered after the patient has fasted for at least 12 hours. In some embodiments, the pharmaceutical tablet composition is administered for at least 12 weeks.

In some embodiments, the administering improves one or more (e.g., two or more) of the following: abdominal pain, abdominal discomfort, abdominal bloating, cramping, abdominal symptom score, IBS symptom severity, treatment satisfaction, and assessment of adequate relief.

Another aspect of the invention is a method of treating or relieving pain comprising administering to a patient in need thereof, a therapeutically effective amount of a pharmaceutical tablet composition as described herein.

In some embodiments, the pain is selected from visceral pain; diverticulitis pain; pelvic pain; abdominal pain; or pain associated with gastrointestinal disorders, venereal diseases, bladder pain syndrome, or interstitial cystitis. In some embodiments, the pain is selected from general abdominal pain, diverticular disease, pain associated with irritable bowel syndrome (IBS), chronic or acute radiation proctopathy (also referred to as radiation proctitis), rectal pain, chronic proctalgia, proctalgia fugax, anal pain, chronic anal fissure, post-operative anal pain, overactive bladder syndrome, stress incontinence, interstitial cystitis, bladder pain syndrome, pain associated with cancer, pain associated with gastrointestinal tract neoplasms, general pelvic pain, endometriosis, orchialgia, chronic prostatitis, prostatodynia, vulvodynia, urethral syndrome, penile pain, perianal pain, and pain associated with ulcerative colitis, ulcerative proctitis, or Crohn's disease.

In some embodiments, the method of treating a patient includes administering a composition comprising a therapeutically effective amount of linaclotide once a day. In some embodiments, the composition is administered once a day in the morning. In some embodiments, the composition is administered once a day at least 30 minutes before ingestion of food. For example, once a day in the morning at least 30 minutes before breakfast. In some embodiments, the composition is administered after the patient has fasted, e.g., after the patient has fasted for at least 2 hours, for at least 4 hours, for at least 8 hours, or for at least 10 hours.

In certain aspects of the present methods, the composition is administered for a period of greater than four weeks, (e.g., at least 8 weeks, at least 12 weeks, or at least 26 weeks). In some aspects of the present method, the linaclotide is administered each day of the week, at least once a week, at least twice a week, at least three times a week, at least four times a week, at least five times a week or at least six times a week.

In another aspect, the method of treating a patient includes administering a delayed release composition comprising a therapeutically effective amount of linaclotide, wherein the administering decreases abdominal pain in the patient. In some embodiments, the abdominal pain is decreased by at least 30% (e.g., at least 40%, at least 50%) compared to a baseline level of abdominal pain prior to treatment with delayed release compositions of linaclotide. In some embodiments, abdominal pain in the patient is decreased compared to treatment with immediate release compositions of linaclotide. In some embodiments, the abdominal pain is decreased by at least at least 30% (e.g., at least 40%, at least 50%) at week 12, after 12 weeks of administration.

In another aspect, the method of treating a patient includes administering a delayed release composition comprising a therapeutically effective amount of linaclotide, wherein the administering improves abdominal symptoms (e.g., pain, discomfort, bloating, cramping) and/or produces no changes in bowel symptoms (e.g., CSBMs/per week, SBMs/per week, stool consistency, straining).

In some embodiments, the method of treating a patient includes treating a disorder selected from irritable bowel syndrome (IBS), irritable bowel syndrome with diarrhea (IBS-d), mixed IBS (IBS-m), un-subtyped IBS (IBS-u), colon cancer, diverticulitis, ulcerative colitis, a functional gastrointestinal disorder, gastroesophageal reflux disease, functional heartburn, dyspepsia, visceral pain, abdominal pain, gastroparesis, chronic intestinal pseudo-obstruction, colonic pseudo-obstruction, Crohn's disease, inflammatory bowel disease, overactive bladder syndrome, bladder hypersensitivity or colitis induced bladder afferent hyperactivity in a patient in need thereof. In some embodiments, the method of treating a patient includes treating a symptom associated with a disorder, such as a GI disorder, or alternatively a non-GI disorder in a patient in need thereof. For example, the treatment may be for abdominal pain, discomfort or bloating, or visceral pain associated with a disorder (GI or non-GI). For example, the patient may be a non-constipated patient.

In further embodiments, the disorder or symptom being treated is selected from visceral pain; diverticulitis pain; pelvic pain; abdominal pain; or pain associated with gastrointestinal disorders, venereal diseases, bladder pain syndrome, or interstitial cystitis. In further embodiments, the disorder or symptom being treated is pain selected from general abdominal pain, diverticular disease, pain associated with irritable bowel syndrome (IBS), chronic or acute radiation proctopathy (also referred to as radiation proctitis), rectal pain, chronic proctalgia, proctalgia fugax, anal pain, chronic anal fissure, post-operative anal pain, overactive bladder syndrome, stress incontinence, interstitial cystitis, bladder pain syndrome, pain associated with cancer, pain associated with gastrointestinal tract neoplasms, general pelvic pain, endometriosis, orchialgia, chronic prostatitis, prostatodynia, vulvodynia, urethral syndrome, penile pain, perianal pain, and pain associated with ulcerative colitis, ulcerative proctitis, or Crohn's disease.

In some embodiments, the disorder or symptom being treated is a disorder or symptom associated with the lower GI (e.g., a lower GI disorder).

In some embodiments, the methods of treatment described herein are useful for the treatment of diseases or symptoms associated with visceral pain selected from the group consisting of general abdominal pain, diverticular disease, pain associated with irritable bowel syndrome (IBS), chronic or acute radiation proctopathy (also referred to as radiation proctitis), rectal pain, chronic proctalgia, proctalgia fugax, anal pain, chronic anal fissure, post-operative anal pain, overactive bladder syndrome, stress incontinence, interstitial cystitis, bladder pain syndrome, pain associated with cancer, pain associated with gastrointestinal tract neoplasms, general pelvic pain, endometriosis, orchialgia, chronic prostatitis, prostatodynia, vulvodynia, urethral syndrome, penile pain, perianal pain, ulcerative colitis, ulcerative proctitis, and Crohn's disease. In one particular embodiment, the compositions described herein are useful for the treatment of bladder pain syndrome. In another particular embodiment, the compositions described herein are useful for the treatment of overactive bladder syndrome (including for example bladder hypersensitivity or colitis induced bladder afferent hyperactivity).

In still another embodiment, the methods of treatment described herein are useful for the treatment of interstitial cystitis. In still another embodiment, the compositions described herein are useful for the treatment of endometriosis. In another embodiment, the compositions described herein are useful for the treatment of anal pain.

In some embodiments, a method of treating a disorder is provided comprising administering to a patient in need thereof, a therapeutically effective amount of the composition described herein. In some embodiments, the disorder is cancer selected from colorectal/local metastasized colorectal cancer, intestinal polyps, Barrett's esophagus, gastrointestinal tract cancer, lung cancer, cancer or pre-cancerous growths or metastatic growths of epithelial cells, polyps, breast, colorectal, lung, ovarian, pancreatic, prostatic, renal, stomach, bladder, liver, esophageal and testicular carcinoma.

In some embodiments, the composition or oral dosage form is administered simultaneously or sequentially with an effective amount of a COX-2 inhibitor. Examples of highly selective and selective COX-2 inhibitors include etoricoxib, rofecoxib, lumiracoxib, valdecoxib, celecoxib (Celebrex®), sulindac, diclofenac, meloxicam and etodolac. Non-selective NSAIDs that inhibit COX-2 include naproxen, ibuprofen, sodium salicylate and diflunisal. As used herein, the term “prevent” or “preventing” means to arrest, delay the onset (i.e., the period prior to clinical manifestation of a disease) or reoccurrence of cancer or hyperplasia, and/or reduce the risk of developing cancer or hyperplasia relative to a patient that has not been treated with a composition described herein.

In some embodiments, a method of treating or relieving pain is provided comprising administering to a patient in need thereof, a therapeutically effective amount of the composition described herein. In some embodiments, the pain is selected from visceral pain; abdominal pain; pelvic pain; or pain associated with gastrointestinal disorders, venereal diseases, bladder pain syndrome, diverticulitis pain, prostatitis, testicular pain, endometriosis, vulvodynia, rectal pain, or interstitial cystitis. In some embodiments, the pain is selected from pelvic pain, pain associated with proctitis, anal fissure pain, pain associated with vulvodynia, pain associated with endometriosis, pain associated with fibromyalgia, functional abdominal pain, interstitial cystitis pain, pain associated with venereal disease, diverticulitis, pain associated with diverticulitis, and pain associated with celiac sprue.

In some embodiments, the effective dose range of linaclotide for adult humans is from 25 μg to 6 mg per day orally. In some embodiments, the dose range is 15 μg to 5 mg per day orally. In some embodiments, the dose range for adult humans is 15 μg to 3 mg per day orally (e.g., 15 μg, 30 μg, 50 μg, 72 μg, 100 μg, 145 μg, 150 μg, 200 μg, 250 μg, 290 μg, 300 μg, 350 μg, 400 μg, 450 μg, 500 μg, 550 μg, 579 μg, 600 μg, 650 μg, 700 μg, 750 μg, 800 μg, 850 μg, 900 μg, 950 μg, 1000 μg, 1200 μg, 1500 μg, or 2000 μg, 2500 μg, or 3000 μg). In some embodiments, the dose range for adult humans is 30 μg to 1200 μg per day orally. In some embodiments, the dose range is 300 μg to 1200 μg per day orally. In some embodiments, the dose is 300 μg, 600 μg, 1200 μg, 200 μg, 300 μg, 400 μg, 500 μg or 600 μg linaclotide per day orally. In some embodiments, the dose is 50 μg linaclotide per day orally. In some embodiments, the dose is 1200 μg linaclotide per day orally. In some embodiments, the dose is 300 μg linaclotide per day orally. In some embodiments, the dose is 500 μg linaclotide per day orally. In some embodiments, the dose is 600 μg linaclotide per day orally. In some embodiments, the dose is 1200 μg linaclotide per day orally. In some embodiments, the dose is 3000 μg linaclotide per day orally.

In some embodiments, the unit dosage form is administered with food at any time of the day, without food at any time of the day, with food after an overnight fast (e.g., with breakfast). In some embodiments, the unit dosage form is administered once a day, twice a day or three times a day. In some embodiments, one, two or three unit dosage forms will contain the daily oral dose of linaclotide. The precise amount of compound administered to a patient will be the responsibility of the attendant physician. However, the dose employed will depend on a number of factors, including the age and sex of the patient, the precise disorder being treated, and its severity.

In some embodiments, the compositions are administered as a monotherapy. In some embodiments, the composition consists essentially of an effective amount of linaclotide. In some embodiments, the composition consists of an effective amount of linaclotide.

In some embodiments, the compositions are directly administered to a patient, for example, in the form of delayed release tablet or delayed release capsule. In some embodiments, the compositions are dissolved, disintegrated and/or mixed on or within food or beverage prior to administration to patients (e.g., elderly or pediatric patients). In some embodiments, the composition is dissolved or disintegrated in a liquid, solution, or fluid optionally containing stabilizing agent(s), preservative(s), sweetener(s), or the like, etc. prior to administration to a patient (e.g., elderly or pediatric patient). In some embodiments, the composition is a multiple dose composition, i.e., containing two, three, five, seven, ten, fifteen, twenty, twenty-five, thirty, forty, fifty, sixty, seventy, eighty, ninety or more daily doses of linaclotide.

In other embodiments, the compositions are administered as part of a combination therapy. For example, a composition may be used in combination with other drugs or therapies that are used in the treatment, prevention, suppression, and/or amelioration of the diseases or conditions for which compounds of the invention are useful. The linaclotide can be co-administered or co-formulated with other medications. In one embodiment, the linaclotide composition can be co-administered with other medications used to treat gastrointestinal disorders including but not limited to acid suppressing agents such as Histamine-2 receptor agonists (H2As) and/or proton pump inhibitors (PPIs). In one embodiment, the linaclotide composition can be co-administered with other medications used to treat gastrointestinal disorders including 5-ASAs such as mesalamine.

Such other drug(s) may be administered, by a route and in an amount commonly used therefore, contemporaneously or sequentially with a compound of the invention. When a compound of the present invention is used contemporaneously with one or more other drugs, a pharmaceutical unit dosage form containing such other drugs in addition to the compound of the invention may be employed. Accordingly, the pharmaceutical compositions of the present invention include those that also contain one or more other active components, in addition to a compound of invention.

Several methods can be used for evaluating the bioactivity of the linaclotide composition, including, but not limited to, immunoassays (e.g., enzyme-linked immunosorbent assay), radioimmuno assays, immunoradiometric assays, gel electrophoresis (e.g., SDS-PAGE), high performance liquid chromatography (HPLC), and/or high performance capillary electrophoresis (HPCE). In some embodiments, the bioactivity of the composition is assessed by a method comprising fixing linaclotide, incubating linaclotide with guanylate cyclase C (GCC), incubating GCC bound linaclotide with antibodies against GCC, incubating GCC antibody-bound linaclotide with fluorescently labeled antibodies against GCC antibodies, and detecting the linaclotide bound to the GCC antibodies by measuring the fluorescence intensity using a plate reader. The drug concentration can then be calculated based on the fluorescence reading of the solution.

For example, the bioactivity of the linaclotide compositions can be assessed and quantified using the following method, though other methods are available. The composition is added to a volumetric flask containing 60 ml of phosphate buffer having a pH of 4.5, and the flask is shaken for 60 minutes. 0.2 ml of the supernatant is then removed, and is added into one or more wells of a 96-well plate that is coated with GC-C receptors. The plate is sealed and incubated at 37° C. for 2 hr. At the end of incubation, the sample is removed and the plate is washed with phosphate buffered saline (PBS). The bound linaclotide is then incubated for 1 hour, at room temperature, with GC-C (such as is available from Sigma-Aldrich Inc.) labeled with fluorescein isocyanate (FITC) in blocking buffer. After incubation, the well is washed with PBS. The fluorescence intensity of the end product is detected, for example, by using a plate reader. The linaclotide concentration is then calculated based on the fluorescence reading of the solution.

B. Delayed Release Compositions

Delayed release oral dosage forms of linaclotide (collectively, “DR”) are provided herein. The delayed release pharmaceutical compositions of the present invention relates to stable, solid, oral dosage forms of linaclotide which exhibit delayed release of linaclotide to the lower gastrointestinal tract. Until now, the only approved formulation of linaclotide is a capsule that exhibits immediate release (“IR”). These IR dosage forms release most or all of the linaclotide contained therein in the upper GI. This, in turn, causes GC-C receptor activation and fluid secretion in both the upper GI and to a lesser extent in the lower GI. The difference between upper and lower GI activation and fluid secretion by the IR dosage form is due, in part, to the fact that linaclotide (once released from the dosage form) undergoes proteolytic digestion and loses some or all capacity to activate GC-C receptors, particularly by the time it reaches the lower GI (such as the ileum, terminal ileum, ileocecal valve, or colon).

In some embodiments, the linaclotide is present in the composition in an amount between 30 μg to 5,000 μg. For example, in some embodiments, the linaclotide is present in an amount of about 300 μg, about 600 μg, about 1200 μg, or about 3,000 μg.

In some embodiments, the composition further comprises between 0%-2% per weight of an amino acid selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, or any mixture thereof. In some embodiments, the composition further comprises between 0.01%-2% or between 0.5%-1.5% per weight of histidine. In some embodiments, the composition further comprises about 1.49% of histidine. In some embodiments, the composition further comprises between 0.01%-3% per weight of a cation salt selected from the group consisting of calcium, potassium, magnesium, zinc, aluminum, manganese, chromium, cobalt, nickel, barium, and sodium, or any combination or mixture thereof. In some embodiments, the composition further comprises between 0.01%-3% per weight of a calcium salt. In some embodiments, the composition further comprises between 0.01%-2% or between 0.2%-0.8% per weight of calcium chloride dehydrate. In some embodiments, the composition further comprises about 0.71% of calcium chloride dehydrate. In some embodiments, the composition further comprises between 0%-5%, between 1%-5%, or between 1%-1.88% per weight of polyvinyl alcohol (PVA). In some embodiments, the composition further comprises about 1.88% or about 3.59% per weight of polyvinyl alcohol (PVA).

In some embodiments, the pH-sensitive polymer has a dissolution pH of at least 6.0, at least 6.5, or at least 7.0. In some embodiments, The pH-sensitive polymer comprises methyl acrylate-methacrylic acid copolymers (e.g., Eudragit®). In some embodiments, the pH-sensitive polymer comprises Eudragit S100. In some embodiments, the pH-sensitive polymer comprises Eudragit L100. In some embodiments, the pH-sensitive polymer consists essentially of Eudragit S100. In some embodiments, the pH-sensitive polymer comprises a mixture of Eudragit S100 and Eudragit L100. In some embodiments, the pH-sensitive polymer comprises a mixture of Eudragit S100 and Eudragit L100 at a ratio of between 1:1 and 6:1 (S100:L100), at a ratio of between 4.5:1 and 5.5:1 (S100:L100), or at a ratio of 4.875:1 (S100:L100) by weight.

In some embodiments, the delayed release pharmaceutical tablet composition comprises an enteric coated tablet. In some embodiments, the delayed release pharmaceutical tablet composition comprises:

Ca²⁺;

histidine; and

polyvinyl alcohol (PVA).

In some embodiments, the composition further comprises a protective polymer film or subcoating. In some embodiments, the subcoating comprises Opadry II®.

The DR dosage forms described herein release most or all of the linaclotide contained therein within the lower GI, such as proximate to the ileocecal valve or within the colon (and less or no release in the stomach, duodenum and/or jejunum). Therefore, the inventive dosage forms have a capacity to achieve lower overall fluid secretion than IR dosage forms in the upper GI, while improving or still maintaining excellent efficacy for treating a disorder (e.g., a GI disorder such as IBS-d or symptoms associated with a disorder, such as abdominal pain). IBS patients report lower left quadrant abdominal pain as a symptom of their disorder, so it is believed that the pain of IBS originates from the colon. Moreover, the DR dosage forms are believed to be ideally suited for treating lower GI-associated diseases and disorders. Because the DR dosage forms will not release any (or a small percentage) of its linaclotide in the stomach and upper GI (which can cause rapid digestion of the linaclotide in the intestine), some preferred embodiments of the DR dosage form will incorporate low doses of linaclotide (as compared to the amounts in the approved IR form) but will maintain the same efficacy levels as the IR in treating GI symptoms. Disorders that are suitable for treatment with the delayed release compositions include irritable bowel syndrome (IBS), irritable bowel syndrome with diarrhea (IBS-d), mixed IBS (IBS-m), un-subtyped IBS (IBS-u), diverticulitis, ulcerative colitis, a functional gastrointestinal disorder, gastroesophageal reflux disease, functional heartburn, dyspepsia, visceral pain, abdominal pain, gastroparesis, chronic intestinal pseudo-obstruction, colonic pseudo-obstruction, Crohn's disease, inflammatory bowel disease, overactive bladder syndrome, bladder hypersensitivity or colitis induced bladder afferent hyperactivity in a patient in need thereof. Symptoms that are suitable for treatment with the delayed release compositions include abdominal pain, discomfort, cramping, or bloating, or visceral pain, for example, in a non-constipated patient.

In general, the guanylate cyclase C (GC-C) receptor is a transmembrane receptor that is located on the apical surface of epithelial cells in the stomach, intestine and lower GI. The receptor has an extracellular ligand-binding domain, a single transmembrane region and a C-terminal guanylyl cyclase domain. When a ligand binds to the extracellular domain of GC-C, the intracellular catalytic domain catalyzes the production of cGMP from GTP. In vivo, this increase in intracellular cGMP initiates a cascade of events that leads to, among other things, increased secretion of chloride and bicarbonate into the intestinal lumen, increased luminal pH, decreased luminal sodium absorption, increased fluid secretion, and acceleration of intestinal transit. cGMP is secreted bi-directionally from the epithelium into the submucosa and lumen. Normally, the pH of the GI tract gradually increases from stomach (pH 1.5-3) to terminal ileum (pH 7-8) before it drops in the colon to pH 5.5-7.0. In addition, there is growing evidence that the potent analgesic effects of linaclotide in vivo are mediated by a pathway linking extracellular cGMP, secreted from IEC (intestinal epithelial cells) into the submucosa following activation of the GC-C/cGMP pathway by linaclotide, to altered function of colonic nociceptors resulting in peripheral analgesia.

Linaclotide binds to the intestinal GC-C receptor which is a regulator of fluid and electrolyte balance in the intestine. Linaclotide is a peptide that consists of the amino acid sequence Cys₁ Cys₂ Glu₃ Tyr₄ Cys₅ Cys₆ Asn₇ Pro₈ Ala₉ Cys₁₀ Thr₁₁ Gly₁₂ Cys₁₃ Tyr₁₄. Any desired form of linaclotide may be used in the composition, for example, any pharmaceutically acceptable salt or hydrate of the peptide, any isolated and/or purified form thereof, or any disulfide form thereof. Linaclotide has disulfide bonds between Cys₁ and Cys₆, Cys₂ and Cys₁₀, and Cys₅ and Cys₁₃.

In some embodiments, the DR composition comprises enteric-coated tablets comprising an immediate release tablet core and containing a unit dose of linaclotide that dissolves only under pH conditions of the distal segment of intestine. In some embodiments, the enteric or functional coating comprises a pH-sensitive polymer.

The pH-sensitive polymer is chosen on the basis of the threshold pH (or dissolution pH) consistent with the pH of the part of the GI tract where release is desired. Therefore, in one embodiment, the enteric coating comprises a pH-sensitive polymer that has a dissolution profile of a pH of at least 6.0, for example, a pH of at least 6.2, a pH of at least 6.4, a pH of at least 6.5, a pH of at least 6.6, a pH of at least 6.8, a pH of at least 7.0, a pH of at least 7.2, a pH of at least 7.4, a pH of at least 7.6 or higher.

In another embodiment, the pH-sensitive polymer is selected from methyl acrylate-methacrylic acid copolymers (e.g. Eudragit®); cellulose acetate succinate (CAS); hydroxy propyl methyl cellulose phthalate (HPMCP); PVA; PVP; PVP-LP, hydroxy propyl methyl cellulose acetate succinate (HPMCAS); polyvinyl acetate phthalate (PVAP); methyl methacrylate-methacrylic acid copolymers; sodium alginate and stearic acid; guar gum; and carbomers. In further embodiments, the enteric coating is selected from Eudragit® FS30D, PlasAcryl®, Eudragit® S100, Eudragit®L100, Eudragit®L100-55, Eudragit® L30D-55, Eudragit® S, Eudragit®RL30D, Eudragit®RS30D, Eudragit® RS, Eudragit® EC, or mixtures thereof. In one embodiment, the pH-sensitive polymer comprises Eudragit S100. In another embodiment, the pH-sensitive polymer comprises Eudragit L100. In still another embodiment, the pH-sensitive polymer consists essentially of Eudragit S100. In still another embodiment, the pH-sensitive polymer comprises a mixture of Eudragit S100 and Eudragit L100. In still another embodiment, the pH-sensitive polymer comprises a mixture of Eudragit S100 and Eudragit L100 at a ratio of between 1:1 and 6:1 (S100:L100) by weight. In another embodiment, the pH-sensitive polymer comprises a mixture of Eudragit S100 and Eudragit L100 at a ratio of between 4.5:1 and 5.5:1 (S100:L100) by weight. In one particular embodiment, the pH-sensitive polymer comprises a mixture of Eudragit S100 and Eudragit L100 at a ratio of 4.875:1 (S100:L100) by weight.

In yet another embodiment, the enteric coating is at least 40 microns in average thickness, for example, at least 45 microns in average thickness, at least 50 microns in average thickness, at least 55 microns in average thickness, at least 60 microns in average thickness, at least 65 microns in average thickness, at least 70 microns in average thickness, at least 75 microns in average thickness, at least 80 microns in average thickness, at least 85 microns in average thickness, at least 90 microns in average thickness, at least 95 microns in average thickness, at least 100 microns in average thickness, at least 105 microns in average thickness, at least 110 microns in average thickness, at least 115 microns in average thickness, or at least 120 microns in average thickness. In another embodiment, the enteric coating has an average thickness of between 55 microns and 100 microns. In still another embodiment, the enteric coating has an average thickness of between 65 microns and 95 microns. In a particular embodiment, the enteric coating has an average thickness of about 75 microns and 85 microns.

In some embodiments, the delayed release composition comprises at least 1.25% (w/w) of PVA, for example, at least 1.49% (w/w) of PVA. In some embodiments, the delayed release composition comprises at least 0.44% (w/w) of CaCl₂, for example, at least 0.71% (w/w) of CaCl₂. In some embodiments, the delayed release composition comprises at least 0.93% (w/w) of histidine, for example, at least 1.49% (w/w) of histidine.

The delayed release compositions may include any effective amount of linaclotide. In some embodiments, for example, the composition comprises from 0.05 μg to 6 mg of linaclotide. In some embodiments, for example, the composition comprises from 1 μg to 5 mg of linaclotide. In some embodiments, the composition comprises from 25 μg to 2 mg of linaclotide, for example, from 50 μg to 1 mg of linaclotide. In some embodiments, for example, the composition comprises from 0.1 μg to 90 μg of linaclotide. In some embodiments, for example, the composition comprises from 0.1 μg to 45 μg of linaclotide. In some embodiments, for example, the composition comprises from 0.1 μg to 25 μg of linaclotide. In some embodiments, for example, the composition comprises from 30 μg to 300 μg of linaclotide. In some embodiments, the composition comprises 0.05 μg, 0.1 μg, 0.15 μg, 0.25 μg, 0.5 μg, 0.75 μg, 1 μg, 1.5 μg, 2 μg, 2.5 μg, 3 μg, 3.5 μg, 4 μg, 4.5 μg, 5 μg, 7.5 μg, 9 μg, 10 μg, 15 μg, 20 μg, 25 μg, 30 μg, 35 μg, 36 μg, 40 μg, 45 μg, 50 μg, 60 μg, 72 μg, 75 μg, 90 μg, 100 μg, 145 μg, 150 μg, 200 μg, 250 μg, 290 μg, 300 μg, 350 μg, 400 μg, 450 μg, 500 μg, 550 μg, 579 μg, 600 μg, 650 μg, 700 μg, 750 μg, 800 μg, 850 μg, 900 μg, 950 μg or 1 mg of linaclotide. In some embodiments, the composition comprises from 100 μg to 600 μg of linaclotide. In some embodiments, the composition comprises 300 μg, 600 μg, 1200 μg, or 3000 μg of linaclotide.

It has been found, in some embodiments, that the stability of delayed release compositions of linaclotide can be increased or improved by including in the compositions a suitable amount of a sterically hindered primary amine (e.g., amino acid) component, a cation (e.g., metal cation) component, and/or a polymer component. These components increase or enhance the stability of delayed release compositions of linaclotide, for example, by preventing, lessening, and/or decreasing degradation of linaclotide within the composition (for example, due to moisture-driven degradation reactions, e.g., hydrolysis, deamidation, and/or multimerization reactions). For instance, it has been found in some embodiments that addition or inclusion of a suitable amount of a cation (e.g., Mg²⁺, Ca²⁺, Zn²⁺) in the composition increases the stability of the composition against oxidative degradation of linaclotide. Moreover, it has been found in some embodiments that inclusion of a suitable amount of a sterically hindered primary amine for an example in the form of an amino acid (e.g., histidine) in the composition increases the stability of the composition against, for example, the nucleophilic addition of formaldehyde or a formaldehyde equivalent to the N-terminus of linaclotide, e.g. by acting as a scavenger, and/or by buffering the composition. Moreover, it has been found in some embodiments that inclusion of both a sterically hindered primary amine (e.g., histidine) and a cation (e.g., Ca²⁺) in suitable amounts in the composition increases the stability of the composition against the formation of hydrolysis and formaldehyde (Cys¹-IMD) products of linaclotide. It has also been found in some embodiments that inclusion of a suitable amount of a polymer (e.g., polyvinyl pyrrolidone or polyvinyl alcohol) in the delayed release composition increases the stability of the composition for example by decreasing the mobility and/or reactivity of linaclotide within the composition, e.g., by forming a complex or matrix (for example, a glassy and/or rigid matrix) with linaclotide (e.g., by vitrification reaction), by preventing or lessening hydrogen bond formation between linaclotide and water molecules, and/or by enhancing the three-dimensional structural integrity of linaclotide.

In this regard, it has been found in some embodiments that combining linaclotide in an delayed release pharmaceutical composition with specific concentrations or molar ratios of the cation and sterically hindered primary amine causes a synergistic enhancement or improvement in the stability of linaclotide within the composition, for example as compared to similar compositions not containing the cation and/or sterically hindered primary amine and/or the same concentrations of these components. In some embodiments, composition can comprise any stabilizing amount of a sterically hindered primary amine component. In other embodiments, the composition comprises a molar ratio of sterically hindered primary amine to linaclotide between 400:1 and 1:1. In further, embodiments, the composition comprises a molar ratio of sterically hindered primary amine to linaclotide between 200:1 and 50:1. In other embodiments, the composition can comprise a molar ratio of sterically hindered primary amine (e.g., amino acid) to linaclotide between 150:1 and 1:100. In some embodiments, the composition comprises a molar ratio of sterically hindered primary amine to linaclotide between 120:1 and 80:1. In some embodiments, the composition comprises a molar ratio of sterically hindered primary amine to linaclotide is about 100:1. In some embodiments, the composition comprises a molar ratio of sterically hindered primary amine to linaclotide between 100:1 and 20:1. In some embodiments, the composition comprises a molar ratio of sterically hindered primary amine to linaclotide between 100:1 and 25:1. In some embodiments, the composition comprises a molar ratio of sterically hindered primary amine to linaclotide between 100:1 and 30:1. In some embodiments, the composition comprises a molar ratio of sterically hindered primary amine to linaclotide between 100:1 and 40:1. In some embodiments, the composition comprises a molar ratio of sterically hindered primary amine to linaclotide between 100:1 and 50:1. In some embodiments, the composition comprises a molar ratio of sterically hindered primary amine to linaclotide between 100:1 and 60:1. In some embodiments, the composition comprises a molar ratio of sterically hindered primary amine to linaclotide between 100:1 and 70:1. In some embodiments, the composition comprises a molar ratio of sterically hindered primary amine to linaclotide of at least 5:1. In some embodiments, the composition comprises a molar ratio of sterically hindered primary amine to linaclotide of at least 10:1. In some embodiments, the composition comprises a molar ratio of sterically hindered primary amine to linaclotide of at least 50:1. In some embodiments, the composition comprises a molar ratio of sterically hindered primary amine to linaclotide of at least 30:1. In some embodiments, the composition comprises a molar ratio of sterically hindered primary amine to linaclotide of at least 40:1.

Suitable sterically hindered primary amines for inclusion in the delayed release composition are, for example, naturally-occurring amino acids (e.g., alanine, arginine, asparagine, aspartic acid, cysteine, glutamic acid, glutamine, glycine, histidine, isoleucine, leucine, lysine, meglumine, methionine, phenylalanine, proline, serine, threonine, tryptophan, tyrosine, valine), synthetic amino acids (e.g., lanthionine, theanine or l-amino cyclohexane), amino sugars (e.g., chitosan or glucosamine), or combination or mixtures thereof. In some embodiments, the composition comprises an amino acid selected from alanine, arginine, asparagine, aspartic acid, cysteine, glutamic acid, glutamine, histidine, isoleucine, leucine, lysine, methionine, phenylalanine, proline, serine, threonine, tryptophan, tyrosine, valine, or a mixture thereof. In some embodiments, the composition comprises an amino acid selected from leucine, isoleucine, asparagine, glutamine, glutamic acid, histidine, cysteine, alanine, serine, threonine, tyrosine, proline, tryptophan, or a combination or mixture thereof. In some embodiments, the composition comprises an amino acid selected from leucine, isoleucine, methionine, alanine, or a combination or mixture thereof. In some embodiments, the composition comprises methionine. In some embodiments, the composition comprises alanine. In some embodiments, the composition comprises histidine.

The delayed release composition can comprise any stabilizing amount of a cation (e.g., metal cation). In some embodiments, the composition comprises a molar ratio of cation to linaclotide between 300:1 and 1:1. In further embodiments, the composition comprises a molar ratio of cation to linaclotide between 250:1 and 30:1. In other embodiments, the composition can comprise a molar ratio of cation to linaclotide between 100:1 and 1:100. In some embodiments, the composition comprises a molar ratio of cation to linaclotide between 100:1 and 1:1. In some embodiments, the composition comprises a molar ratio of cation to linaclotide between 90:1 and 2:1. In some embodiments, the composition comprises a molar ratio of cation to linaclotide between 80:1 and 5:1. In some embodiments, the composition comprises a molar ratio of cation to linaclotide between 70:1 and 10:1. In some embodiments, the composition comprises a molar ratio of cation to linaclotide between 60:1 and 20:1. In some embodiments, the composition comprises a molar ratio of cation to linaclotide between 50:1 and 30:1. In some embodiments, the composition comprises a molar ratio of cation to linaclotide is about 50:1. In some embodiments, the composition comprises a molar ratio of cation to linaclotide between 100:1 and 25:1. In some embodiments, the composition comprises a molar ratio of cation to linaclotide between 80:1 and 30:1. In some embodiments, the composition comprises a molar ratio of cation to linaclotide between 60:1 and 40:1. In some embodiments, the composition comprises a molar ratio of cation to linaclotide of at least 5:1. In some embodiments, the composition comprises a molar ratio of cation to linaclotide of at least 10:1. In some embodiments, the composition comprises a molar ratio of cation to linaclotide of at least 20:1. In some embodiments, the composition comprises a molar ratio of cation to linaclotide of at least 25:1. In some embodiments, the composition comprises a molar ratio of cation to linaclotide of at least 30:1. In some embodiments, the composition comprises a molar ratio of cation to linaclotide of at least 40:1.

Any suitable cation(s) can be included in the composition, for example, any suitable metal cation or organic cation. In some embodiments, the composition comprises a metal cation selected from calcium, potassium, magnesium, zinc, aluminum, iron, tin, manganese, chromium, cobalt, nickel, barium, sodium, or a combination or mixture thereof. In some embodiments, the composition comprises a metal cation selected from calcium, potassium, magnesium, zinc, aluminum, manganese, chromium, cobalt, nickel, barium, sodium, or a combination or mixture thereof. In some embodiments, the composition comprises a metal cation selected from aluminum, calcium, potassium, sodium, magnesium, manganese, zinc, or a combination or mixture thereof. In some embodiments, the composition comprises a metal cation selected from calcium, magnesium, manganese, zinc, or a combination or mixture thereof. In some embodiments, the composition comprises a divalent metal cation. In some embodiments, the composition comprises a divalent metal cation selected from Al³⁺, Ca²⁺, Mg²⁺, Zn²⁺, Mn²⁺, or a combination or mixture thereof. In some embodiments, the composition comprises Mg²⁺. In some embodiments, the composition comprises Ca²⁺. In some embodiments, the composition comprises Zn²⁺. In some embodiments, the composition comprises Al³⁺.

Moreover, the metal cation can be added to the composition in any suitable form, for example any pharmaceutically acceptable salt with any appropriate counterion. Suitable metal salts include, for example, calcium chloride, calcium carbonate, calcium acetate, magnesium chloride, magnesium acetate, zinc acetate, zinc chloride, or mixtures thereof. In some embodiments, the composition comprises calcium chloride, magnesium chloride, zinc acetate, or any combination or mixture thereof. In some embodiments, the composition comprises calcium chloride. In some embodiments, the composition comprises magnesium chloride. In some embodiments, the composition comprises zinc acetate. Suitable organic cations include, for example, ammonium hydroxide, D-arginine, L-arginine, t-butylamine, calcium acetate hydrate, calcium carbonate, calcium DL-malate, calcium hydroxide, choline, ethanolamine, ethylenediamine, glycine, L-histidine, L-lysine, magnesium hydroxide, N-methyl-D-glucamine, L-omithine hydrochloride, potassium hydroxide, procaine hydrochloride, L-proline, pyridoxine, L-serine, sodium hydroxide, DL-tryptophan, tromethamine, L-tyrosine, L-valine, carnitine, taurine, creatine malate, arginine alpha ketoglutarate, omithine alpha ketoglutarate, spermine acetate, spermidine chloride, or combinations or mixtures thereof. In some embodiments, the organic cation is selected from the group consisting of N-methyl D-glucamine, choline, arginine, lysine, procaine, tromethamine (TRIS), spermine, N-methyl-morpholine, glucosamine, N,N-bis(2-hydroxyethyl) glycine, diazabicycloundecene, creatine, arginine ethyl ester, amantadine, rimantadine, omithine, taurine, and citrulline, or any combination or mixture thereof.

The composition can contain any stabilizing amount of a polymer. In some embodiments, the composition comprises between 1 and 25% by weight of a polymer, relative to the total weight of the composition. In some embodiments, the composition comprises between 1 and 10% by weight of a polymer, relative to the total weight of the composition.

In some embodiments, the composition comprises between 2 and 4% by weight of a polymer, relative to the total weight of the composition. In some embodiments, the composition comprises between 0.01 and 5 wt. % of a polymer. In some embodiments, the composition comprises between 0.1 and 4 wt. % of a polymer. In some embodiments, the composition comprises about 0.71 wt. % of a polymer. In some embodiments, the composition comprises about 3.59 wt. % of a polymer.

In some embodiments, the polymer acts as both a stabilizer, protective coating, or as a film forming agent within the delayed release composition. In some embodiments, the delayed release composition comprises a molar ratio of polymer (e.g., PVP or PVA) to linaclotide between 80:1 and 300:1, for example, between 100:1 and 200:1, between 110:1 and 190:1, or even between 120:1 and 180:1. In some embodiments, the delayed release composition comprises a molar ratio of polymer (e.g., PVP or PVA) to linaclotide greater than about 80:1, for example, greater than about 100:1, or even greater than about 120:1. In some embodiments, the delayed release composition comprises a weight ration of polymer (e.g., PVP or PVA) to linaclotide between 10:1 and 300:1, for example, between 80:1 and 200:1, between 100:1 and 180:1, or even between 110:1 and 150:1. In some embodiments, the delayed release composition comprises a weight ration of polymer (e.g., PVP or PVA) to linaclotide between 100:1 and 500:1, for example, between 200:1 and 400:1, between 250:1 and 350:1, or even between 300:1 and 350:1.

Suitable polymers for inclusion in the delayed release compositions are, for example, polyvinyl pyrrolidone (PVP), polyvinyl alcohol (PVA), polyvinyl alcohol low peroxide (PVA-LP), hydroxylpropyl methyl cellulose (HPMC), hydroxylpropyl cellulose (HPC), methyl cellulose, methacrylate polymers, cyclodextrin, dextrin, dextran, polyacrylic acid, chitosan, guar gum, xanthan gum, polyethylene oxide (e.g., polyethylene polypropylene oxide), poly (sodium vinylsulfonate), polyethylene glycol, poly(arginine), poly carbophil, polyvinyl pyrrolidone-co-vinyl acetate, a poloxamer (e.g., Pluronic® products available from BASF), alginate, trehalose, sucrose, inulin, or a combination or mixture thereof. In some embodiments, the composition comprises a polymer selected from PVP, PVA, methacrylate polymers, cyclodextrin, dextran, polyacrylic acid, chitosan, guar gum, xanthan gum, polyethylene oxide, polyethylene glycol, poly(arginine), poly carbophil, polyvinyl pyrrolidone-co-vinyl acetate, a poloxamer, or a combination or mixture thereof. In some embodiments, the composition comprises PVP, PVA, polyethylene oxide, or a mixture thereof. In some embodiments, the composition comprises PVP, PVA, or a mixture thereof. In some embodiments, the composition comprises PVP. In some embodiments, the composition comprises PVA.

In some embodiments, the composition comprises two or more stabilizing agents. For example, the composition can include a stabilizing amount of a polymer and a stabilizing amount of a sterically hindered primary amine. Moreover, the composition can include a stabilizing amount of a polymer and a stabilizing amount of a cation (e.g., metal cation). In addition, the composition can include a stabilizing amount of a sterically hindered primary amine and a stabilizing amount of a cation (e.g., metal cation). In some embodiments, the composition comprises a stabilizing amount of a polymer, a stabilizing amount of a sterically hindered primary amine, and a stabilizing amount of a cation (e.g., metal cation).

In some embodiments, the delayed release composition comprises a stabilizing amount of PVP and a stabilizing amount of an amino acid selected from histidine, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or a mixture thereof. In some embodiments, the composition comprises a stabilizing amount of PVP and a stabilizing amount of an amino acid selected from alanine, arginine, asparagine, aspartic acid, cysteine, glutamic acid, glutamine, histidine, isoleucine, leucine, lysine, methionine, phenylalanine, proline, serine, threonine, tryptophan, tyrosine, valine, or a mixture thereof. In some embodiments, the composition comprises a stabilizing amount of PVP and a stabilizing amount of histidine.

In some embodiments, the delayed release composition comprises a stabilizing amount of PVP and a stabilizing amount of a cation (e.g., metal cation). In some embodiments, the composition comprises a stabilizing amount of PVP and a stabilizing amount of a divalent metal cation. In some embodiments, the composition comprises a stabilizing amount of PVP and a stabilizing amount of Mg²⁺, Ca²⁺, Zn²⁺ or a salt thereof or a combination or mixture thereof. In some embodiments, the composition comprises a stabilizing amount of PVP and a stabilizing amount of Ca²⁺ or a salt thereof. In some embodiments, the composition comprises a stabilizing amount of PVP and a stabilizing amount of Mg²⁺ or a salt thereof. In some embodiments, the composition comprises a stabilizing amount of PVP and a stabilizing amount of Zn²⁺ or a salt thereof.

In some embodiments, the delayed release composition comprises a stabilizing amount of an amino acid selected from histidine and a stabilizing amount of a divalent metal cation selected from Mg²⁺, Ca²⁺, Zn²⁺ or a salt thereof or a combination or mixture thereof. In some embodiments, the delayed release composition comprises (i) a stabilizing amount of PVP or PVA, (ii) a stabilizing amount of histidine, and (iii) a stabilizing amount of Mg²⁺, Ca²⁺, Zn²⁺ or a salt thereof or a combination or mixture thereof.

In some embodiments, the composition comprises (i) between 0.1 and 30 wt. % of a polymer, (ii) a sterically hindered primary amine (e.g., an amino acid) in a molar ratio of primary amine to linaclotide between 150:1 and 10:1, and (iii) a cation (e.g., a metal cation) in a molar ratio of cation to linaclotide between 60:1 and 40:1.

The delayed release composition (e.g., delayed release tablet) may also comprise any one or more filling agents. Suitable filling agents include, but are not limited to, starch, calcium carbonate, calcium sulfate, hydroxylpropylmethyl cellulose, fructose, methyl cellulose, dextrates, dextrose, dextran, lactitol, maltose, sucrose, sorbitol, isomalt, pregelatinized starch, dicalcium phosphate, microcrystalline cellulose, mannitol, gelatin, trehalose, erythritol, maltitol, lactose, glucose, or a combination thereof, or a mixture thereof. In some embodiments, the filling agent is isomalt. In some embodiments, the filling agent is gelatin. In some embodiments, the filling agent is mannitol. In some embodiments, the filling agent is pregelatinized starch. In some embodiments, the filling agent is microcrystalline cellulose.

The delayed release composition (e.g., delayed release tablet) can comprise any suitable concentration of filling agent. In some embodiments, for example, the composition comprises one or more filling agents in a concentration of 0.1-99% by weight, relative to the total weight of the composition. In some embodiments, for example, the composition comprises one or more filling agents in a concentration of 1-95 wt. % of filling agent(s), relative to the total weight of the composition. In some embodiments, for example, the composition comprises one or more filling agents in a concentration of 10-90 wt. % of filling agent(s), relative to the total weight of the composition. In some embodiments, for example, the composition comprises one or more filling agents in a concentration of 20-90 wt. % of filling agent(s), relative to the total weight of the composition. In some embodiments, for example, the composition comprises one or more filling agents in a concentration of 25-85 wt. % of filling agent(s), relative to the total weight of the composition. In some embodiments, for example, the composition comprises one or more filling agents in a concentration of 30-80 wt. % of filling agent(s), relative to the total weight of the composition. In some embodiments, for example, the composition comprises one or more filling agents in a concentration of 40-70 wt. % of filling agent(s), relative to the total weight of the composition. In some embodiments, for example, the composition comprises one or more filling agents in a concentration of 10-60 wt. % of filling agent(s), relative to the total weight of the composition. In some embodiments, for example, the composition comprises one or more filling agents in a concentration of 20-50 wt. % of filling agent(s), relative to the total weight of the composition. In some embodiments, the composition comprises one or more filling agents in a concentration of at least 20 wt. %, for example, at least 40 wt. %, at least 60 wt. %, at least 70 wt. %, at least 80 wt. %, or at least 90 wt. %, relative to the total weight of the composition.

In some embodiments, the delayed release composition (e.g., delayed release film) comprises one or more plasticizers. Suitable plasticizers include, but are not limited to, polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the concentration of the plasticizer in the formulation may be about 0 to about 30 wt. %, for example, about 1 to about 20 wt. %, about 0 to about 10 wt. %, about 1 to about 5 wt. %, or even 0 to about 4 wt. %.

In some embodiments, the delayed release composition comprises a film forming agent, a water-soluble polymer, a pH sensitive polymer, biodegradable polymer, or combination thereof. Water soluble, pH sensitive, or biodegradable polymers that may be used in the orally dissolving formulations of the present invention include, but are not limited to, cellulose derivatives, synthetic polymers polyacrylates and natural gums. For example, the water soluble polymers used in the orally dissolving formulations of the present invention may include, but are not limited to, methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phthalate, cellulose acetate butyrate, amylose, dextran, casein, pullulan, gelatin, pectin, agar, carrageenan, xanthan gum, tragacanth, guar gum, acacia gum, arabic gum, polyethylene glycol, polyethylene oxide, polyvinyl pyrrolidone, polyvinyl alcohol, cyclodextrin, carboxyvinyl polymers, sodium alginate, polyacrylic acid, methylmethacrylate or mixtures thereof. In exemplary embodiments, the concentration of the water-soluble polymer in the formulation may be about 20% to about 90% (by weight), preferably between about 40% to about 80% (by weight).

In some embodiments, the pH sensitive polymer is Eudagrit® L100 that has a threshold pH (also called dissolution pH) of 6.0. In some embodiments, the pH sensitive polymer is Eudagrit® 5100 that has a threshold pH of 7.0. In some embodiments, the pH sensitive polymer is Eudagrit® L-30D that has a threshold pH of 5.6. In some embodiments, the pH sensitive polymer is Eudagrit® FS 30D that has a threshold pH of 6.8. In some embodiments, the pH sensitive polymer is Eudagrit® L100-55 that has a threshold pH of 5.5. In some embodiments, the pH sensitive polymer is Polyvinyl acetate phthalate that has a threshold pH of 5.0. In some embodiments, the pH sensitive polymer is Hydroxypropylmethylcellulose phthalate that has a threshold pH of 4.5-4.8. In some embodiments, the pH sensitive polymer is Hydroxypropylmethylcellulose phthalate 50 that has a threshold pH of 5.2. In some embodiments, the pH sensitive polymer is Hydroxypropylmethylcellulose phthalate 55 that has a threshold pH of 5.4. In some embodiments, the pH sensitive polymer is Cellulose acetate trimelliate that has a threshold pH of 4.8. In some embodiments, the pH sensitive polymer is Cellulose acetate phthalate that has a threshold pH of 5.0. In some embodiments the delayed release composition comprises a combination of the pH sensitive polymers mentioned above.

One skilled in the art, with the benefit of this disclosure, will understand that other components may be included to enhance one or more properties of the delayed release composition. In some embodiments, for example, the delayed release compositions may include one or more disintegrants, lubricants, anti-caking additives, anti-microbial agents, antifoaming agents, emulsifiers, surfactants, buffering agents, and/or coloring agents.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof. In some embodiments, the disintegrant is crospovidone. In some embodiments, the disintegrant is croscarmellose sodium.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL® 200, W.R. Grace Co., Baltimore, Md. USA), a coagulated aerosol of synthetic silica (Evonik Degussa Co., Plano, Tex. USA), a pyrogenic silicon dioxide (CAB-O-SIL, Cabot Co., Boston, Mass. USA), and mixtures thereof.

Suitable anti-caking additives include, for example, calcium silicate, magnesium silicate, silicon dioxide, colloidal silicon dioxide, talc, glyceryl, and mixtures thereof.

Suitable anti-microbial additives that may be used, e.g., as a preservative for the linaclotide compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymol, and mixtures thereof.

The composition may also comprise any suitable pharmaceutically acceptable carrier or medium. Suitable pharmaceutically acceptable carriers include, for example, any solvents, dispersants, pH buffering agents, coatings, absorption promoting agents, controlled release agents, and one or more inert excipients (e.g., filling agents, starches, polyols, granulating agents, microcrystalline cellulose, diluents, lubricants, binders, disintegrating agents), or the like. In addition, the compositions can contain any desired additional components, additives, and/or species, for example, surface active additives, dispersing additives, humectants, suspending agents, solubilizers, buffering agents, disintegrants, preservatives, colorants, flavorants, and the like. In some embodiments, the composition comprises one or more ion species that interact with linaclotide.

In some embodiments, there is provided a pharmaceutical composition comprising linaclotide, and one or more peptides selected from:

i. a peptide (“Cys¹-IMD”) or a pharmaceutically acceptable salt thereof, wherein the peptide comprises the amino acid structure of:

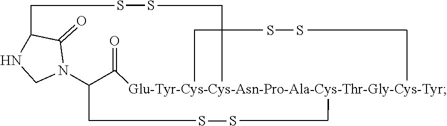

ii. a hydrolysis peptide (“Asp”) or a pharmaceutically acceptable salt thereof, wherein the peptide comprises the amino acid structure of:

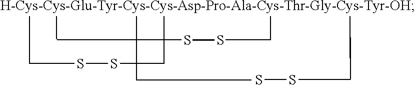

iii. an acetylation peptide (“Cys¹-N-Acetyl”) or a pharmaceutically acceptable salt thereof, wherein the peptide comprises the amino acid structure of:

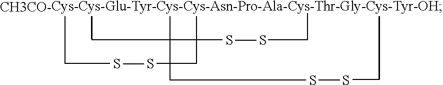

iv. a linaclotide trisulfide peptide or a pharmaceutically acceptable salt thereof, wherein the peptide comprises the amino acid sequence of Cys Cys Glu Tyr Cys Cys Asn Pro Ala Cys Thr Gly Cys Tyr wherein an additional sulfur atom may be attached to any one of the six cysteinyl sulfurs;
v. a peptide (“Des-Tyr¹⁴”) or a pharmaceutically acceptable salt thereof, wherein the peptide comprises the amino acid structure of:

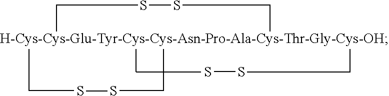

or
vi. a peptide (Cys¹-α-Ketone) or a pharmaceutically acceptable salt thereof, wherein the peptide comprises the amino acid structure of:

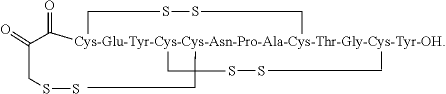

In some embodiments, thea Cys¹-α-Ketone peptide may be present in its hydrated form or a pharmaceutically acceptable salt thereof, wherein the peptide comprises an amino acid structure of:

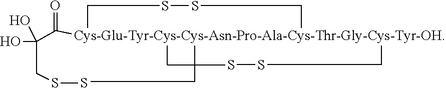

One skilled in the art would recognize that the Cys¹-α-Ketone peptide would readily convert between its hydrate and ketone form.

In some embodiments, the Cys¹-α-Ketone peptide comprises less than about 15% by weight of the composition, less than about 10% by weight of the composition, less than about 7% by weight of the composition, less than about 5% by weight of the composition, less than about 4% by weight of the composition, less than about 3% by weight of the composition, less than about 2% by weight of the composition, less than about 1.5% by weight of the composition, or less than about 1% by weight of the composition. In other exemplary embodiments, the Cys¹-α-Ketone peptide comprises from about 0.01% to about 15% by weight of the composition, about 0.05% to about 10% by weight of the composition, about 0.05% to about 7% by weight of the composition or about 0.05% to about 5% by weight of the composition.

In some embodiments, the Cys¹-IMD peptide comprises less than about 15% by weight of the composition, less than about 10% by weight of the composition, less than about 7% by weight of the composition, less than about 5% by weight of the composition, less than about 4% by weight of the composition, less than about 3.5% by weight of the composition, less than about 3% by weight of the composition, less than about 2% by weight of the composition, or less than about 1% by weight of the composition. In other exemplary embodiments, the Cys¹-IMD peptide comprises from about 0.01% to about 15% by weight of the composition, about 0.05% to about 10% by weight of the composition, about 0.05% to about 7% by weight of the composition or about 0.05% to about 5% by weight of the composition.

In some embodiments, the hydrolysis peptide (“Asp⁷”) comprises less than about 15% by weight of the composition, less than about 10% by weight of the composition, less than about 7% by weight of the composition, less than about 5% by weight of the composition, less than about 4% by weight of the composition, less than about 3.5% by weight of the composition, less than about 3% by weight of the composition, less than about 2% by weight of the composition, or less than about 1% by weight of the composition. In other exemplary embodiments, the hydrolysis peptide (“Asp⁷”) comprises from about 0.01% to about 15% by weight of the composition, about 0.05% to about 10% by weight of the composition, about 0.05% to about 7% by weight of the composition or about 0.05% to about 5% by weight of the composition.

In some embodiments, the acetylation peptide (“Cys¹-N-Acetyl”) comprises less than about 15% by weight of the composition, less than about 10% by weight of the composition, less than about 7% by weight of the composition, less than about 5% by weight of the composition, less than about 4% by weight of the composition, less than about 3.5% by weight of the composition, less than about 3% by weight of the composition, less than about 2% by weight of the composition, or less than about 1% by weight of the composition. In other exemplary embodiments, the acetylation peptide (“Cys¹-N-Acetyl”) comprises from about 0.01% to about 15% by weight of the composition, about 0.05% to about 10% by weight of the composition, about 0.05% to about 7% by weight of the composition or about 0.05% to about 5% by weight of the composition.

In some embodiments, the linaclotide trisulfide peptide comprises less than about 15% by weight of the composition, less than about 10% by weight of the composition, less than about 7% by weight of the composition, less than about 5% by weight of the composition, less than about 4% by weight of the composition, less than about 3.5% by weight of the composition, less than about 3% by weight of the composition, less than about 2% by weight of the composition, or less than about 1% by weight of the composition. In other exemplary embodiments, the linaclotide trisulfide peptide comprises from about 0.01% to about 15% by weight of the composition, about 0.05% to about 10% by weight of the composition, about 0.05% to about 7% by weight of the composition or about 0.05% to about 5% by weight of the composition.

In some embodiments, the Des-Tyr¹⁴ peptide comprises less than about 15% by weight of the composition, less than about 10% by weight of the composition, less than about 7% by weight of the composition, less than about 5% by weight of the composition, less than about 4% by weight of the composition, less than about 3.5% by weight of the composition, less than about 3% by weight of the composition, less than about 2% by weight of the composition, or less than about 1% by weight of the composition. In other exemplary embodiments, the Des-Tyr¹⁴ peptide comprises from about 0.01% to about 15% by weight of the composition, about 0.05% to about 10% by weight of the composition, about 0.05% to about 7% by weight of the composition or about 0.05% to about 5% by weight of the composition.

In some embodiments, the composition comprises linaclotide and any desired concentration of multimers. In some embodiments, the composition comprises less than 10 wt. % of multimer(s). In some embodiments, the composition comprises between 0.5 and 1 wt. % of multimer(s).

In some embodiments, the composition comprises an effective amount of linaclotide and any desired amount of reduced form linaclotide. As used herein, the term “reduced form linaclotide” refers to linaclotide having no disulfide bonds between cysteine amino acids. In some embodiments, the composition comprises less than 10 wt. % of reduced form linaclotide. In some embodiments, the composition comprises between 0.5 and 1 wt. % of reduced form linaclotide.

In some embodiments, the composition comprises an effective amount of linaclotide and any desired amount of scrambled form linaclotide. As used herein, the term “scrambled form linaclotide” refers to linaclotide having disulfide bonds between Cys₁ and Cys₁₀, between Cys₁ and Cys₁₃, between Cys₁ and Cys₅, between Cys₁ and Cys₂, between Cys₂ and Cys₆, between Cys₂ and Cys₁₃, between Cys₂ and Cys₅, between Cys₅ and Cys₆, and/or between Cys₅ and Cys₁₀. In some embodiments, the composition comprises between 0.5 and 1 wt. % of scrambled form linaclotide. In some embodiments, the composition comprises less than 10 wt. % of scrambled form linaclotide.

In some embodiments, the composition comprises a total degradant concentration of less than about 10 wt. %. In some embodiments, the composition comprises a total degradant concentration of less than about 8 wt. %. In some embodiments, the composition comprises a total degradant concentration of less than about 7 wt. %. In some embodiments, the composition comprises a total degradant concentration of less than about 6.5 wt. %. In some embodiments, the composition comprises a total degradant concentration of less than about 6 wt. %. In some embodiments, the composition comprises a total degradant concentration of less than about 5.5 wt. %. In some embodiments, the composition comprises a total degradant concentration of less than about 5 wt. %. In some embodiments, the composition comprises a total degradant concentration of less than about 4 wt. %. In some embodiments, the composition comprises a total degradant concentration of less than about 3 wt. %. In some embodiments, the composition comprises a total degradant concentration of less than about 2.5 wt. %. In some embodiments, the composition comprises a total degradant concentration of less than about 2 wt. %. In some embodiments, the composition comprises a total degradant concentration of less than about 1 wt. %.

In some embodiments, the compositions can be prepared by spray drying, which is a technique used to prepare microparticles (e.g., microcapsules or microspheres) of drugs. Spray-dried peptides generally retain their biological activity upon dissolution and may have useful physical characteristics, including a uniform particle size and a spherical shape. In addition, the microparticles prepared by spray drying are often free flowing, which is helpful for pharmaceutical manufacturing processes such as forming tablets and filling capsules. Spray drying processes are also useful because they may be readily scaled up for clinical and commercial manufacturing. In one embodiment, the spray buffer comprises HCl, histidine, 1.5% PVA and 0.6% talc. This formulation can be used to produce lower dosing ranges between 30-1200 μg.

The composition, when administered, will dissolve to release linaclotide in targeted areas of the gastrointestinal tract. The formulation may release the linaclotide over a period of time that is determined by a number of different factors. These factors include the dimensions of the formulation, the concentration of the linaclotide, and how the linaclotide is dispersed throughout the formulation. For example, by varying the thickness and surface area of the formulations the rate of dissolution may be adjusted. A thick formulation will dissolve more slowly than an otherwise similar thin formulation and may be desirable to administer high dosages of linaclotide.

In some embodiments, the delayed release composition has a disintegration rate of less than about 60 minutes in the targeted pH conditions. In some embodiments, the delayed release composition has a disintegration rate of less than about 30 minutes in the targeted pH conditions. In some embodiments, the delayed release composition has a disintegration rate of less than about 25 minutes. In some embodiments, the delayed release composition has a disintegration rate of less than about 20 minutes. In some embodiments, the delayed release composition has a disintegration rate of less than about 15 minutes. In some embodiments, the delayed release composition has a disintegration rate of less than about 10 minutes. In some embodiments, the delayed release composition disintegrates in less than about 30 minutes after entering a targeted environment. In some embodiments, the delayed release composition disintegrates in less than about 25 minutes after entering a targeted environment. In some embodiments, the delayed release composition disintegrates in less than about 20 minutes after entering a targeted environment. In some embodiments, the delayed release composition disintegrates in less than about 15 minutes after entering a targeted environment.

In some embodiments, the delayed release composition releases at least about 75% of the linaclotide contained therein within 60 minutes of entering a targeted environment. In some embodiments, the delayed release composition releases at least about 75% of the linaclotide contained therein within 30 minutes of entering a targeted environment. In some embodiments, the delayed release composition releases at least about 80% of the linaclotide contained therein within 30 minutes of entering a targeted environment. In some embodiments, the delayed release composition releases at least about 85% of the linaclotide contained therein within 30 minutes of entering a targeted environment. In some embodiments, the delayed release composition releases at least about 90% of the linaclotide contained therein within 30 minutes of entering a targeted environment. In some embodiments, the delayed release composition releases at least about 95% of the linaclotide contained therein within 30 minutes of entering a targeted environment. In some embodiments, the delayed release composition releases at least about 99% of the linaclotide contained therein within 30 minutes of entering a targeted environment.

In some embodiments, the delayed release composition releases at least about 40% of the linaclotide contained therein within 15 minutes of entering a targeted environment. In some embodiments, the delayed release composition releases at least about 50% of the linaclotide contained therein within 15 minutes of entering a targeted environment. In some embodiments, the delayed release composition releases at least about 60% of the linaclotide contained therein within 15 minutes of entering a targeted environment. In some embodiments, the delayed release composition releases at least about 70% of the linaclotide contained therein within 15 minutes of entering a targeted environment. In some embodiments, the delayed release composition releases at least about 80% of the linaclotide contained therein within 15 minutes of entering a targeted environment. In some embodiments, the delayed release composition releases at least about 85% of the linaclotide contained therein within 15 minutes of entering a targeted environment. In some embodiments, the delayed release composition releases at least about 90% of the linaclotide contained therein within 15 minutes of entering a targeted environment. In some embodiments, the delayed release composition releases at least about 95% of the linaclotide contained therein within 15 minutes of entering a targeted environment.

In some embodiments, the delayed release composition releases at least about 80% of the linaclotide contained therein between about 2 minutes to about 2 hours of entering a targeted environment.

In some embodiments, the delayed release composition releases at least about 75% of the linaclotide contained therein within 30 minutes of contacting a pH greater than 5. In some embodiments, the delayed release composition releases at least about 80% of the linaclotide contained therein within 30 minutes of contacting a pH greater than 5. In some embodiments, the delayed release composition releases at least about 85% of the linaclotide contained therein within 30 minutes of contacting a pH greater than 5. In some embodiments, the delayed release composition releases at least about 90% of the linaclotide contained therein within 30 minutes of contacting a pH greater than 5. In some embodiments, the delayed release composition releases at least about 95% of the linaclotide contained therein within 30 minutes of contacting a pH greater than 5. In some embodiments, the delayed release composition releases at least about 99% of the linaclotide contained therein within 30 minutes of contacting a pH greater than 5.

In some embodiments, the delayed release composition releases at least about 75% of the linaclotide contained therein within 30 minutes of contacting a pH greater than 7. In some embodiments, the delayed release composition releases at least about 80% of the linaclotide contained therein within 30 minutes of contacting a pH greater than 7. In some embodiments, the delayed release composition releases at least about 85% of the linaclotide contained therein within 30 minutes of contacting a pH greater than 7. In some embodiments, the delayed release composition releases at least about 90% of the linaclotide contained therein within 30 minutes of contacting a pH greater than 7. In some embodiments, the delayed release composition releases at least about 95% of the linaclotide contained therein within 30 minutes of contacting a pH greater than 7. In some embodiments, the delayed release composition releases at least about 99% of the linaclotide contained therein within 30 minutes of contacting a pH greater than 7.

In some embodiments, the linaclotide DR compositions are formulated for delivery of linaclotide to the ileum, late ileum, or colon. In some embodiments, the linaclotide DR compositions are formulated for delivery of linaclotide to the ileum or ileal region. In some embodiments, the linaclotide DR compositions are formulated for delivery of linaclotide to within the late ileum to the ascending colon (e.g., at or near the ileocecal junction).

In some embodiments, the composition or oral dosage form is administered to a pediatric patient in need thereof as a tablet, capsule or sachet. In some embodiments, a sachet comprising the composition is opened and the contents are sprinkled on or stirred into food, such as applesauce, or into a beverage, such as water. In some embodiments, a capsule is swallowed whole with fluid, such as water, or is opened and sprinkled on or stirred into food or a beverage. Tablets may be swallowed whole, may be crushed and stirred into food or a beverage, or may be formulated as a chewable tablet.

A subject or patient in whom administration of the pharmaceutical composition is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions described herein are particularly suited for administration to any animal, particularly a mammal, and including, but by no means limited to, humans, rodents and non-rodents, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

In some embodiments, the linaclotide composition may be formulated as a rectal dosage form for rectal administration. Rectal dosage forms include, without limitation, rectal suppositories, rectal foams or aerosols, enemas, rectal gels and rectal ointments. In some embodiments, the rectal dosage form may be administered to a patient in need thereof. In some embodiments, the rectal dosage form may be administered to a pediatric or geriatric patient.

Another aspect of the invention is a method of making a delayed release composition, the method comprising: preparing a linaclotide base, pregranulated filler, and placebo base; and blending and compressing the linaclotide base, pregranulated filler, and placebo base into a tablet. In some embodiments, the pregranulated filler is prepared through wet granulation and dried before blending and compressing into a tablet. In some embodiments, the method further comprises applying a subcoat to the tablet. In some embodiments, the method further comprises applying an enteric or functional coating to the tablet.

Another aspect of the invention is a method of making a delayed release composition, the method comprising: preparing an aqueous solution comprising linaclotide, or a pharmaceutically acceptable salt thereof and applying the aqueous solution to a pharmaceutically acceptable carrier.

### Definitions

As used herein, unless otherwise indicated, the term “delayed release” mean that the composition dissolves, melts, disintegrates, liquefies, etc. in a targeted area of the gastrointestinal tract such that substantially all of the linaclotide no longer remains in a formulation, composition, or dosage form. Delayed release compositions include sustained release compositions, gastro-retentive compositions, targeted release compositions (e.g. colonic-release compositions, or compositions that target the ileosecal valve, etc.), extended release compositions and/or combinations thereof.

As used herein, unless otherwise indicated, the term “delayed release composition” (“DR”) means that the composition is a dosage form that releases linaclotide at a time other than immediately following oral administration.

As used herein, unless otherwise indicated, the term “extended release composition” means that the composition is a dosage form that releases linaclotide over an extended period of time after administration. This allows a reduction in dosing frequency compared to immediate release compositions.

As used herein, unless otherwise indicated, the “disintegration” and “release” is used herein to mean that the capsule, film, bead, or tablet comprising linaclotide dissolves, melts, disintegrates, liquefies, etc. in the environment of an oral cavity such that substantially all of the linaclotide no longer remains in a formulation form, e.g., a pH greater than 5 or 7, or in a phosphate buffer solution and maintained at 37±1° C.

The term “released from”, when referring to the release of linaclotide from the composition, unless otherwise indicated, is used herein to mean that the linaclotide no longer remains in a composition form.

As used herein, unless otherwise indicated, the term “entry into a targeted environment” means contact of the composition within a patient at a targeted organ or segment thereof, or within a segment of the GI intended for linaclotide release, e.g., having a pH greater than 5 or 7.

As used herein, unless otherwise indicated, the term “lower gastrointestinal (GI)” means the distal segment of the gastrointestinal tract, for example, the ileum, terminal ileum, ileocecal valve, or colon.

As used herein, unless otherwise indicated, the term “upper gastrointestinal (GI)” means the proximate segment of the gastrointestinal tract, for example, the stomach, duodenum and/or jejunum.

As used herein, unless otherwise indicated, “stabilizing agent” refers to a polymer, sterically hindered primary amine (e.g., amino acid), or cation (e.g., metal cation) component of the composition which is included in the composition in a stabilizing amount. For example, a polymeric stabilizing agent is a polymer that is included in the composition in a stabilizing amount. Similarly, a sterically hindered primary amine stabilizing agent is a sterically hindered primary amine that is included in the composition in a stabilizing amount. Moreover, a cationic stabilizing agent is a cation that is included in the composition in a stabilizing amount.

As used herein, unless otherwise indicated, “stabilizing amount” refers to a concentration, within the composition, of a polymer, sterically hindered primary amine (e.g., amino acid), or metal cation component at which the component increases the stability of linaclotide in the composition, as compared to a similar composition not having a stabilizing amount of the same component.

As used herein, unless otherwise indicated, the term “substantially all” means at least about 90%, for example, at least about 95% or even at least about 99%.

As used herein, unless otherwise indicated, the term “isolated and purified” means at least 95 percent pure (for example, at least 96% pure, at least 97% pure, at least 98% pure, or even at least 99% pure), as measured, for example, by chromatographic purity using HPLC.

As used herein, unless otherwise indicated, “therapeutically effective amount” means the amount of a linaclotide or a pharmaceutically acceptable salt thereof that, when administered to a mammal for treating a state, disorder or condition, is sufficient to effect a treatment (as defined below). The “therapeutically effective amount” will vary depending on the compound, the disease and its severity and the age, sex, weight, physical condition and responsiveness of the mammal to be treated. For example, a therapeutically effective amount of linaclotide, or its pharmaceutically acceptable salt or hydrate, can be an amount effective to treat gastrointestinal disorders, including irritable bowel syndrome with constipation.

As used herein, unless other indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used herein, unless otherwise indicated, the term “treat”, in all its verb forms, is used herein to mean to relieve, alleviate, prevent, and/or manage at least one symptom of a disorder in a subject, the disorder including, for example, a gastrointestinal disorder, such as irritable bowel syndrome with constipation. Within the meaning of the present invention, the term “treat” also denotes, to arrest, delay the onset (i.e., the period prior to clinical manifestation of a disease) and/or reduce the risk of developing or worsening a disease. The term “treatment” means the act of “treating” as defined above.

As used herein, unless otherwise indicated, the term “prevent” refers to the prophylactic treatment of a subject who is at risk of developing a condition (e.g., stress related disorder) resulting in a decrease in the probability that the subject will develop the condition.

As used herein, unless otherwise indicated, the term “adverse event” refers to any untoward medical occurrence in a patient or clinical investigation subject administered a pharmaceutical product and which does not necessarily have a causal relationship with the treatment. For example, one adverse event is diarrhea.

As used herein, unless otherwise indicated, the term “additives” refers to a pharmaceutically acceptable additive. Pharmaceutically acceptable additives include, without limitation, binders, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, unless otherwise indicated, an “excipient” is any pharmaceutically acceptable additive, filler, binder or agent.

As used herein, unless otherwise indication, “stressed conditions” refer to 40° C. and 75% relative humidity (RH).

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

All weight percentages (i.e., “% by weight” and “wt. %” and w/w) referenced herein, unless otherwise indicated, are measured relative to the total weight of the pharmaceutical composition.

The term “consisting essentially of”, and variants thereof, when used to refer to the composition, are used herein to mean that the composition includes linaclotide and other desired pharmaceutically inactive additives, excipients, and/or components (e.g., polymers, sterically hindered primary amines, cations, filling agents, binders, carriers, excipients, diluents, disintegrating additives, lubricants, solvents, dispersants, coating additives, absorption promoting additives, hydrolysis products, formaldehyde imine products, oxidation products, acetylation products, deamidation products, multimers, controlled release additives, anti-caking additives, anti-microbial additives, preservatives, sweetening additives, colorants, flavors, desiccants, plasticizers, dyes, or the like), and no other active pharmaceutical ingredient(s).

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

Enteric-coated tablets comprising a core immediate release tablet containing a unit dose of linaclotide can be coated with coatings that dissolve only under pH conditions of the distal segment of intestine, so that linaclotide will be released in lower GI tract.

Linaclotide or a pharmaceutically acceptable salt thereof may be produced and purified using standard techniques known in the art, e.g., chemical synthesis or recombinant expression followed by purification using standard techniques.

Preparation of the linaclotide coating solution for beads: Approximately 32 g to 42 g of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The pH of the solution is tested, and hydrochloric acid is added, if necessary, to produce a solution having a pH between 1.5 and 2.0. The binder is then added to the solution and the mixture is then stirred for sufficient time to achieve a clear solution. The desired amount of linaclotide is added to the solution and mixed for 30-100 minutes to provide the coating solution.

In one embodiment, the coating solution comprises linaclotide, histidine, 1.5% PVA and 0.6% talc. This formulation can be used to produce dosing ranges between 30-300 μg.

Preparation of the Active Beads: Approximately 30-36 g of dried microcrystalline cellulose beads are added to a Mini Column Fluid Bed Coater. The microcrystalline cellulose beads are fluidized and heated prior to layering. Next, the coating solution is layered to the beads. The spraying temperature is controlled between 24° C. and 55° C. by controlling inlet temperature, spray rate, atomization pressure, and air volume. After the entire coating solution is layered to the beads, the beads are dried. The product of this process is referred to as active beads.

### Example 1

### Delayed Release Linaclotide Tablet

Linaclotide can be formulated into a tablet for delayed drug release. Compared to an equal volume of beads, tablets have much smaller specific surface area, which makes them potentially less prone to degradation induced by environmental factors such as humidity, oxidation, deamidation, etc. In addition, the smaller surface area of the tablet can become advantageous when an enteric coating is needed since much less coating material is required to cover the surface of the dosage form.

Enteric coatings may be applied in a tablet coating pan, and coatings that are used for delayed release beads can be used for tablets to form delayed release tablets. The amount of coating polymer on the tablet can vary from 5 to 60% (weight gain) depending on the size, shape and surface properties of the tablet. A sub-coat can be applied to the tablets to separate linaclotide from the enteric or functional coat.

### Example 2

### Enteric Coated Tablet

TABLE 1
Eudragit ® FS30D Coated linaclotide
delayed release (DR) tablet composition

| | | |
|---|---|---|
| Ingredients | Wt. % | Wt. in kg |

Fluid bed Granulation

| | | | |
|---|---|---|---|
| 1 | Linaclotide | 0.3 | 2.94 |
| 2 | Isomalt | 93.7 | 937 |
| 3 | Histidine | 0.46 | 4.6 |
| 4 | Calcium chloride dihydrate | 2.57 | 25.7 |
| 5 | Polyvinyl pyrrolidone (PVP) | 3 | 30 |
| 6 | 0.01N HCl | Q.S. | Q.S. |
| 7 | Purified Water | Q.S. | Q.S. |

Blending and compression

| | | | |
|---|---|---|---|
| i | Linaclotide granules | 27.85 | 139.25 |
| ii | Isomalt | 60.9 | 304.5 |
| iii | Crospovidone | 10 | 50 |
| iv | Magnesium stearate | 0.75 | 3.75 |
| vi | Talc | 0.5 | 2.5 |

Enteric coating

| | | |
|---|---|---|
| Linaclotide tablet | 75.19 | 1000 |
| Eudragit ® FS 30D | 22.56 | 1000 |
| PlasACRYL ™ | 2.25 | 150 |
| Purified Water* | — | 500 |
| Total Dry weight | 100 | 1330 |

### Manufacturing Process:

### A. Tablet

The granulation solution may be prepared by dissolving PVP, histidine and calcium chloride in water, adjusting solution pH to 2, and dissolving linaclotide. Granulation is performed in a fluid bed by spraying the granulation solution onto filler isomalt. At the end of granulation, dry the granules for 30 min. The granules are then blended with tablet components including isomalt, crospovidone, Mg stearate and talc until uniform, and compressed into tablets.

### B. Enteric Coating

For tablet coating, linaclotide core tablets are placed into a pan coater and warmed up to 35° C. Start tablets coating with Eudragit® FS 30 D suspension, keep the product temperature at 28° to 32° C., and atomization air pressure at 3 bar. At the end of coating, discharge the tablets and place them into a circulated air oven and dry for 2 h at 40° C. Similarly, other enteric coatings such as Eudragit® L, S, ethyl cellulose, HPMCAS, PVAP, CAP, CAS, etc. may also be applied to form delayed release tablets at various weight gains.

### Example 3

### Delayed Release Compositions Comprising Linaclotide

Delayed release capsules comprising linaclotide may be formulated to target the ileum or colon (e.g., the ileum, late ileum, and/or ascending colon). The composition is formulated to include a pH triggered release based on enteric coating of a linaclotide tablet, capsule or linaclotide coated beads contained in a hard gelatin capsule. The composition may be formulated to further comprise stabilizing additives such as a divalent cation and an amino acid. PVA can be used as binder as well as protective layer in between linaclotide and enteric coating. Linaclotide or linaclotide with PVA overcoat (as beads, capsule or tablet) may be coated with an additional enteric coating (e.g. Eudragit® FS30D, Eudragit® 5100, Eudragit® L100, Eudragit®L100-55, Eudragit® L 30D-55) that dissolves in a pH dependent manner to release at the appropriate pH of 7 in the ileum of the GI tract. The enteric coatings may consist of blends combining different types of Eudragit®—Eudragit® 5100/Eudragit® L100 in different ratios (e.g. 50/50 ratio); Eudragit® 5100/Eudragit® L100-55 in various ratios; Eudragit® FS30D/Eudragit® L 30D-55, Eudragit® FS30DEudragit® S/Eudragit® RS or EC in various ratios. The compositions may further comprise other excipients including plasticizing agents such as triethylcitrate. The coatings may further comprising disintegrants as suspended solid to expedite the relevant pH triggered release—resulting in mixed systems as croscarmellose sodium/Eudragit® S. For ease of processing, anti-tacking agent (e.g., talc, Aerosil® 200 or PlasAcryl™) may be used to prevent the beads from sticking.

Additionally, two Eudragit® coatings may be applied to ensure swift release once the desired pH region in the GI tract is reached—including partially neutralized coating systems. Buffering agents such as potassium hydrogen phosphate can be included into one of the two Eudragit® films. Alternative non-Eudragit® pH dependent film coatings include hydroxypropylmethylcellulose acetate succinate (HPMCAS, e.g. Aqoat® AS-HF), cellulose acetate phthalate (CAP, e.g. Aquateric®) or shellac.

### Example 4

### Measurement of Content and Purity of Exemplary Peptides

Linaclotide, immidazolidinone degradant product (“Cys¹-MD”), and α-ketone degradant product (“Cys¹-α-Ketone”) can be measured and purified as described in US 2010/0048489, US 2013/0190238, and US 2015/0094272 which are incorporated by reference herein. Generally, content and purity of linaclotide may be determined by reverse phase gradient liquid chromatography using an Agilent Series 1100 LC System with Chemstation Rev A.09.03 software or equivalent. A YMC Pro™ C18 column (dimensions: 3.0×150 mm, 3.5 um, 120 Å; Waters Corp., Milford, Mass.) or equivalent is used and is maintained at 40° C. Mobile phase A (MPA) consists of water with 0.1% trifluoroacetic acid while mobile phase B (MPB) consists of 95% acetonitrile:5% water with 0.1% trifluoroacetic acid. Elution of the linaclotide is accomplished with a gradient from 0% to 47% MPB in 28 minutes followed by a ramp to 100% MPB in 4 minutes with a 5 minute hold at 100% MPB to wash the column. Re-equilibration of the column is performed by returning to 0% MPB in 1 minute followed by a 10 minute hold at 100% MPA. The flow rate is 0.6 mL/min and detection is accomplished by UV at 220 nm.

### Example 5

### Linaclotide Tablet Preparation

Delayed release tablets may be prepared by first preparing the following core tablet components: a placebo base, a linaclotide 750 μg/225 mg base, and pre-granulated fillers.

### Granulation Manufacturing Process:

The tablet components may be prepared into separate granulations for blending before tablet compression. Use of separate tablet components, such as, the placebo base and pregranulated filler base provided, among other things, advantageous properties for stability and release profiles for the tablets. For example, all the tablets components listed in Table 2 could be separately prepared by wet granulation and blended before compression or blended together and processed as a mixture for wet granulation. In another process, the tablets components listed in Table 2 could be separately prepared by dry granulation and blended before compression or blended together and processed as a mixture for dry granulation. In another process, the tablet components are direct blended for compression. In a preferred process, the pregranulated filler base and/or placebo base are prepared through wet granulation and dried before mixing with the 750 μg/225 mg linaclotide base. The linaclotide base could be prepared by wet granulation processes or by Wurster coating process. This preferred process, exhibited further gains in stability for the tablet by reducing moisture exposure to linaclotide during processing and minimizing residue moisture in the tablet core.

TABLE 2
Components for various tablet strengths

| Strength | Placebo | 25 μg | 30 μg | 50 μg | 75 μg | 100 μg | 150 μg | 290 μg | 300 μg |
|---|---|---|---|---|---|---|---|---|---|
| Placebo base (%) | 20.00 | 16.67 | 16 | 13.33 | 10.00 | 6.67 | 3.33 | 0.00 | 0 |
| Linaclotide | 0.00 | 3.33 | 4 | 6.67 | 10.00 | 13.33 | 16.67 | 38.65 | 40 |
| base (i.e.750 | | | | | | | | | |
| ug/225 mg | | | | | | | | | |
| base (%)) | | | | | | | | | |
| Pregranulated | 78.75 | 78.75 | 78.75 | 78.75 | 78.75 | 78.75 | 78.75 | 60.10 | 58.75 |
| fillers (%) | | | | | | | | | |
| Magnesium | 1.25 | 1.25 | 1.25 | 1.25 | 1.25 | 1.25 | 1.25 | 1.25 | 1.25 |
| Stearate (%) | | | | | | | | | |
| Total (%) | 100.0 | 100.0 | 100 | 100.0 | 100.0 | 100.0 | 100.0 | 100.0 | 100 |

Then compress the above blends on a suitable tablet press to target core tablet weight of 225 mg. In a perforated pan coater, add a sub-coat (OPADRY® II) at a weight gain of 4% w/w. Coating conditions should be set and monitored so that moisture uptake during coating is kept to a minimum. When measured by loss on drying (LOD), the sub-coated tablets should have no more than 1.5% LOD. In a perforated pan coater, add a functional coat on the subcoated tablets. The functional coat is either Eudragit® FS30D, Eudragit® 5100, or Eudragit® L100. Apply the functional coat at 5 mg polymer weight/cm² of the tablet surface. This comes to be approximately 4.5% total polymer weight gain during functional coating. Coating conditions should be set and monitored so that moisture uptake during coating is kept to a minimum. When measured by loss on drying, the functionally coated tablet should have no more than 2.0% LOD.

### Placebo Base Preparation:

Table 3 represents the formulation for the placebo base granulation:

TABLE 3
Formulation of the placebo base granulation

| Component | % w/w | Quantity (g) |
|---|---|---|
| L-Histidine | 2.26 | 112.9 |
| Calcium Chloride Dihydrate | 1.07 | 53.5 |
| polyvinyl alcohol | 1.50 | 75.0 |
| microcrystalline cellulose | 95.17 | 4758.6 |
| Hydrochloric acid, pure, fuming, | | |
| 37% solution in water | | |
| Treated water | | |
| Total | 100 | 5000 |

The placebo base preparation may be prepared by first dispensing the raw materials of Table 4.

TABLE 4
Raw materials for placebo base preparation

| Component | Quantity (g) |
|---|---|
| L-Histidine | 242.2 |
| Calcium Chloride Dihydrate | 114.8 |
| polyvinyl alcohol | 160.9 |
| microcrystalline cellulose | 4758.6 |

Tare the mix container and add 2682.1±5.0 g of treated water into the container. Set up a mixer and begin to stir the water. Add the EMPROVE® to the water while stirring and start the timer. Cover and heat solution to 70 C while stirring and maintain temperature until material is visually dissolved.

Adjust the pH of solution to 1.5 with hydrochloric acid. Add calcium chloride dihydrate to the solution while stirring. Mix until dissolved. Add L-Histidine to the solution while stirring. Stir for approximately 15 minutes. Record the initial pH. Adjust pH of solution to 5.0 with hydrochloric acid. Record final pH of solution and hydrochloric acid addition. Mix until all material is dissolved. While mixing, adjust the pH solution to 2.5 with hydrochloric acid. Record final pH of solution and hydrochloric acid addition. Ensure that the high shear granulator is set up properly for granulating with the 25 L bowl, mixing blade and chopper. Pass microcrystalline cellulose through 16 mesh screen into granulator bowl. Calculate the net weight of granulation solution to add. Pump the granulation solution into the granulator at a rate of approximately 300 g/min while mixing with the below parameters: Impeller speed 1 (290 rpm, 5.5 m/s tip speed), Chopper speed 1 (1760 rpm). Stop the granulator and scrape down the sides and the bottom of the bowl. Mix for an additional 3 minutes according to the following parameters: Impeller speed 1 (290 rpm, 5.5 m/s tip speed), Chopper speed 1 (1760 rpm). Tare a poly bag and discharge the completed wet granulation into it. Weigh the granulation. Transfer the wet granulation to the FLM-3 fluid bed for drying. Dry the granulation using the following approximate settings. Dry until the granulation LOD is no more than 1.2% moisture. Discharge the dried granulation into a tared poly bag.

TABLE 5
Drying Parameters

| Parameters | Target Range | |
|---|---|---|
| Product Temperature | 40° | C. |
| Process (Drying) Air | 30-60 | CFM |
| Inlet Temperature | 60° | C. |

The settings provided in Table 5 are suggested settings only and may be adjusted for optimum drying.

Screen the dried granulation through a #30 mesh sieve. Tare a poly bag and discharge the dried granulation into it. Weigh the granulation. Package dried granulation into foil sealed bags with desiccant.

Linaclotide Base Preparation (i.e. 750 μg/225 mg):

Table 6 represents the formulation for the 750 μg/225 mg base granulation:

TABLE 6
Formulation for the 750 μg/225 mg base granulation

| | % | |
|---|---|---|
| Component | w/w | Quantity (g) |
| Linaclotide | 0.39 | 19.3 |
| L-Histidine | 2.26 | 112.9 |
| Calcium Chloride Dihydrate | 1.07 | 53.5 |
| polyvinyl alcohol | 1.50 | 75.0 |
| microcrystalline cellulose | 94.79 | 4,739.3 |
| Hydrochloric acid, pure, fuming, | — | — |
| 37% solution in water | | |
| Treated water | — | — |
| Total | 100.00 | 5,000.0 |

The 750 μg/225 mg base granulation may be prepared by first dispensing the raw materials of Table 7.

TABLE 7
Raw materials of the linaclotide base granulation

| | Required |
|---|---|
| Raw Material | Quantity (g) |
| Linaclotide | 19.3 |
| microcrystalline | 4,739.3 |
| cellulose | |
| Granulation solution | |

While mixing, add the linaclotide to the granulation solution. Mix until dissolved. Ensure that the high shear granulator is set up properly for granulating with the 25 L bowl, mixing blade and chopper. Pass microcrystalline cellulose through 16 mesh screen into granulator bowl. Pump the granulation solution into the granulator at a rate of approximately 300 g/min while mixing with the below parameters: Impeller speed 1 (290 rpm, 5.5 m/s tip speed), Chopper speed 1 (1760 rpm). Tare a poly bag and discharge the completed wet granulation into it. Weigh the granulation. Transfer the wet granulation to the fluid bed for drying. Dry the granulation using the following approximate settings. Dry until the granulation LOD is no more than 1.2% moisture as provided in Table 8. Discharge the dried granulation into a tared poly bag.

TABLE 8
Drying Parameters

| Parameters | Target Range | |
|---|---|---|
| Process (Drying) Air | 30-60 | CFM |
| Inlet Temperature | 80° | C. |

Note:
Settings are suggested settings only and may be adjusted for optimum drying.

Screen the dried granulation through a #30 mesh sieve. Tare a poly bag and discharge the dried granulation into it. Weigh the granulation. Package dried granulation into foil sealed bags with desiccant.

### Pregranulated Filler Preparation:

Table 9 represents the formulation for the pregranulated fillers.

TABLE 9
Formulation of the fillers granulation

| | % | Qty per sublot |
|---|---|---|
| Component | w/w | (g) |
| microcrystalline | 19.4 | 1,358 |
| cellulose | | |
| croscarmellose sodium | 5.1 | 357 |
| mannitol | 71.7 | 5,019 |
| polyvinyl alcohol | 3.8 | 266 |
| Treated water | — | — |
| Total | 100.0 | 7,000 |

The fillers preparation may be prepared by first dispensing the raw materials of Table 10.

TABLE 10
Raw materials for preparation of fillers granulation

| | Required |
|---|---|
| Raw Material | Quantity (g) |
| polyvinyl alcohol | 266 |
| microcrystalline cellulose | 1,358 |
| croscarmellose sodium | 357 |
| mannitol | 5,019 |
| Total | — |

Then record the tare weight of the stainless steel container. Tare the container and weigh the required quantity of treated water into the container. Transfer the water into a jacketed kettle. Set up the mixer and begin to stir the water in the kettle. Add the EMPROVE® (polyvinyl alcohol) to the water while stirring and start the timer. Cover and heat solution to 70° C. while stirring and maintain temperature until material is visually dissolved. Calculate weight of water lost due to evaporation during heating. Add this amount of treated water to the solution. Add each of microcrystalline cellulose, croscarmellose sodium), and mannitol to a high shear granulator bowl. Mix for approximately 2 minutes according to the following parameters: Impeller speed 1 (290 rpm, 5.5 m/s tip speed), Chopper speed 1 (1760 rpm). Pump 2217±5 g of the granulation solution into the granulator at a rate of approximately 300 g/min while mixing with the below parameters: Impeller speed 1 (290 rpm, 5.5 m/s tip speed), Chopper speed 1 (1760 rpm). Stop the granulator and scrape down the sides and the bottom of the bowl. Mix for an additional 30 seconds to 1 minute according to the following parameters: Impeller speed 1 (290 rpm, 5.5 m/s tip speed), Chopper speed 1 (1760 rpm). Tare a poly bag and discharge the completed wet granulation into it. Weigh the granulation. Pass the wet granulation through the Comil with 2A375Q03763 screen with 5-10% power. Transfer the wet granulation to the FLM-3 fluid bed for drying. Dry the granulation using the following approximate settings. Dry until the granulation LOD is no more than 1.0% moisture as provided in Table 11. Discharge the dried granulation into a tared poly bag.

TABLE 11
Drying Parameters

| Parameters | Target Range |
|---|---|
| Process (Drying) Air | 30-60 CFM |
| Inlet Temperature | 80° C. |

Note:
Settings are suggested settings only and may be adjusted for optimum drying.

Mill the granulation with Comil, round impeller, 2A045R03137 screen. Tare a poly bag and discharge the dried and milled granulation into it. Weigh the granulation. Package dried granulation into foil sealed bags with desiccant.

### Example 6

### Alternative Functional or Enteric Coating of Linaclotide Tablets (“DR2”)

An alternative coating, may be prepared according to the method of Example 5 but with the formulation of Table 12. The coating of this Example 6 is used as a coating for the DR2 delayed release compositions discussed herein.

TABLE 12
Formulation for functional coating process

| Component | % w/w |
|---|---|
| Eudragit ® S100 | 9.94 |
| 1N NH₃ | 6.75 |
| Triethyl Citrate | 4.97 |
| Talc | 4.97 |
| Purified water | 73.37 |
| Total | 100 |

### Example 7

### Organic Coating of Linaclotide Tablets

An organic coating may be provided for the linaclotide tablets of the examples above. For the coating of 100 μg tablets, the formula of Table 13 was used.

TABLE 13
Organic coating material formula for linaclotide tablets.

| Component | % w/w | g/batch |
|---|---|---|
| Eudragit S100 | 2.941 | 88.2 |
| Eudragit L100 | 2.941 | 88.2 |
| Triethyl Citrate | 1.177 | 35.3 |
| Talc | 2.941 | 88.2 |
| Acetone | 34.290 | 1028.7 |
| Isopropanol | 51.420 | 1542.6 |
| Purified Water | 4.290 | 128.7 |
| Total | 100 | 3000 |

Dispense the required quantity of purified water into a suitable sized container. Dispense the required quantity of acetone into a suitable sized container. Begin mixing acetone and add the water. Dispense the required quantity of isopropanol into a suitable sized container. Add isopropanol to the water and acetone to create the diluent mixture. Pour approximately half of the diluent mixture into a second container and resume mixing the first half of the diluent mixture. Dispense the required quantity of Eudragit S100 into a suitable sized container. Begin mixing the second half of diluent mixture and add the Eudragit S100. Dispense the required quantity of Eudragit L100 into a suitable sized container.

Add the Eudragit L100 to the diluent mixture containing the Eudragit S100 and start timer. Stir until the polymers are completely dissolved. Add the triethyl citrate to the first half of the diluent mixture while mixing with a high shear mixer. Dispense the required quantity of talc into a suitable sized container. Add the talc to the first half of the diluent mixture while mixing with a high shear mixer to create the excipient suspension. Start the timer and mix for at least 10 minutes. Record the mixing time of the Eudragit solution. While continuing to mix the Eudragit solution, slowly pour the excipient suspension into the Eudragit solution. Once the coating suspension is uniform pass it through a 35 mesh screen. Resume mixing. Calculate the theoretical amount of solution needed to apply a 8.5% weight gain, with 88% theoretical efficiency.

Prepare a poly bag for the waste tablets collected during the coating process. Ensure the Compu-Lab has been set up with the 15 inch pan and plenum assembly. Verify the liquid feed lines are solvent resistant 17 tubing. Verify the gun assembly is installed in the pan. The spray gun assembly should consist of 1×¼ JAU spray gun mounted with a 40100 AB liquid spray nozzle and matching air cap. The gun assembly should be mounted as far as possible from the tablet bed, with the spray angle perpendicular to the top third of the bed. Adjust the pump so that the liquid flow rate is approximately 28 g/min. Prime the lines past the guns and verify that there is no leaking in the lines or gun. Charge the tablets into the coating pan and begin warming with an inlet temperature of 38° C. and airflow of 200 CFM. Jog occasionally during warm-up. Once the product temperature reaches about 27° C., begin spraying the coating suspension according to the target process parameters outlined below. When 8.5% weight gain has been achieved, stop spray and dry tablets for 10 minutes with an inlet temperature of 40° C., reducing pan speed to a minimum or jogging. These exemplary processing parameters are summarized in Table 14.

TABLE 14
Processing Parameters

| Parameters | Target |
|---|---|
| Spray rate | 27-30 g/min |
| Inlet temperature | 30-40° C. |
| Airflow | 200 CFM |
| Atomization air | 17 PSI |
| Pan speed | 13 RPM |
| Exhaust temperature | 25-28° C. |
| Bed Temperature | 26-27° C. |

Place tablets onto trays and dry for at least 24 hours in a mechanical convection oven with the temperature set to 45° C.

### Example 8

### Alternative Linaclotide Tablet Preparation

Delayed release tablets were prepared by first preparing the following core tablet components: a placebo base, a linaclotide 1000 μg/225 mg base, and pre-granulated fillers.

### Granulation Manufacturing Process:

The tablet components were prepared essentially as described above in Example 7, but with slight modifications as described below. The placebo base as described in Table 15 was used to provide core tablets with the components listed in Table 15. A final pH of 2.25 was used for placebo and active base granulations.

TABLE 15
Components for various tablet strengths

| | | 25 | 30 | 50 | 75 | 100 | 150 | 290 | 300 |
|---|---|---|---|---|---|---|---|---|---|
| Strength | Placebo | μg | μg | μg | μg | μg | μg | μg | μg |
| Placebo base (%) | 30 | 27.5 | 27 | 25 | 22.5 | 20 | 15 | 1 | 0 |
| Linaclotide | 0 | 2.5 | 3 | 5 | 7.5 | 10 | 15 | 29 | 30 |
| base (i.e., | | | | | | | | | |
| 1000 ug/225 | | | | | | | | | |
| mg base (%)) | | | | | | | | | |
| Pregranulated | 68.75 | 68.75 | 68.75 | 68.75 | 68.75 | 68.75 | 68.75 | 68.75 | 68.75 |
| fillers (%) | | | | | | | | | |
| Magnesium | 1.25 | 1.25 | 1.25 | 1.25 | 1.25 | 1.25 | 1.25 | 1.25 | 1.25 |
| Stearate (%) | | | | | | | | | |
| Total (%) | 100 | 100 | 100 | 100 | 100 | 100 | 100 | 100 | 100 |

### Placebo Base Preparation:

Table 16 represents the formulation for the placebo base granulation:

TABLE 16
Formulation of the placebo base granulation

| Component | % w/w | Quantity (g) |
|---|---|---|
| L-Histidine | 4.97 | 248.5 |
| Calcium Chloride Dihydrate | 2.35 | 117.5 |
| polyvinyl alcohol | 3.96 | 198 |
| microcrystalline cellulose | 88.72 | 4436 |
| Hydrochloric acid, pure, fuming, | | |
| 37% solution in water | | |
| Treated water | | |
| Total | 100 | 5000 |

The placebo base preparation was prepared by first dispensing the raw materials of Table 17.

TABLE 17
Raw materials for placebo base preparation

| Component | Quantity (g) |
|---|---|
| L-Histidine | 248.4 |
| Calcium Chloride Dihydrate | 117.7 |
| polyvinyl alcohol | 198 |
| microcrystalline cellulose | 4435.9 |

The mix container is tared and 2200±5.0 g of treated water (rather than 2682.1±5.0 g) is added into the container.

Linaclotide Base Preparation (1000 μg/225 mg):

Table 18 represents the formulation for the 1000 μg/225 mg base granulation:

TABLE 18
Formulation for the 1000 μg/225 mg base granulation

| | % | |
|---|---|---|
| Component | w/w | Quantity (g) |
| Linaclotide | 0.444 | 23 |
| L-Histidine | 4.97 | 248.4 |
| Calcium Chloride Dihydrate | 2.35 | 117.7 |
| polyvinyl alcohol | 3.96 | 198 |
| microcrystalline cellulose | 88.27 | 4413.7 |

A balance of 0.02% represents Linaclotide impurities.

The 1000 μg/225 mg base granulation may be prepared by first dispensing the raw materials of Table 19.

TABLE 19
Raw materials of the linaclotide base granulation

| | Required |
|---|---|
| Raw Material | Quantity (g) |
| Linaclotide | 23 |
| microcrystalline | 4413.7 |
| cellulose | |
| Granulation solution | |

### Pregranulated Filler Preparation:

Table 20 represents the formulation for the pregranulated fillers.

TABLE 20
Formulation of the fillers granulation

| | % | Qty per sublot |
|---|---|---|
| Component | w/w | (g) |
| microcrystalline | 14.6 | 1018 |
| cellulose | | |
| croscarmellose sodium | 2.9 | 203.6 |
| mannitol | 79.1 | 5533.2 |
| polyvinyl alcohol | 3.5 | 245 |
| Treated water | — | — |
| Total | 100.0 | 7,000 |

The fillers preparation are prepared by first dispensing the raw materials of Table 21.

TABLE 21
Raw materials for preparation of fillers granulation

| | Required |
|---|---|
| Raw Material | Quantity (g) |
| polyvinyl alcohol | 245 |
| microcrystalline cellulose | 1018 |
| croscarmellose sodium | 203.6 |
| mannitol | 5533.2 |
| Total | — |

Table 22 provides alternative 225 mg tablet formulations for Linaclotide at 30 μg, 100 μg, and 300 μg loadings.

TABLE 22
Alternative 225 mg tablet formulation (A = DR2)

| | % w/w | | | | | |
|---|---|---|---|---|---|---|
| | 30 μg strength | | 100 μg strength | | 300 μg strength | |
| Component | A | B | A | B | A | B |

Tablet Core

| | | | | | | |
|---|---|---|---|---|---|---|
| linaclotide^{a} | 0.013 | 0.013 | 0.044 | 0.044 | 0.132 | 0.132 |
| L-Histidine | 0.5 | 1.49 | 0.5 | 1.49 | 0.9 | 1.49 |
| Calcium Chloride | 0.2 | 0.71 | 0.2 | 0.71 | 0.4 | 0.71 |
| Dihydrate | | | | | | |
| Polyvinyl Alcohol | 3.3 | 1.88 | 3.3 | 1.88 | 2.7 | 1.88 |
| MCC PH 200^{a} | 34.3 | 41.73 | 34.3 | 41.70 | 49.3 | 41.61 |
| Mannitol 100SD | 56.5 | 47.94 | 56.5 | 47.94 | 42.1 | 47.94 |
| Crosscarmellose | 4.00 | 5.00 | 4.00 | 5.00 | 3.0 | 5.00 |
| sodium | | | | | | |
| Magnesium | 1.25 | 1.25 | 1.25 | 1.25 | 1.25 | 1.25 |
| Stearate | | | | | | |
| Purified Water | —^{b} | —^{b} | —^{b} | —^{b} | —^{b} | —^{b} |
| Total (Tablet | 100.0 | 100.0 | 100.0 | 100.0 | 100.0 | 100.0 |
| Core) | | | | | | |

Barrier Coat Suspension

| | | | | | | |
|---|---|---|---|---|---|---|
| Opadry II White | 4 ^{c} | 4 ^{c} | 4 ^{c} | 4 ^{c} | 4 ^{c} | 4 ^{c} |
| 85F18422 | | | | | | |
| Purified Water | —^{b} | —^{b} | —^{b} | —^{b} | —^{b} | —^{b} |

Functional Coat Suspension

| | | | | | | |
|---|---|---|---|---|---|---|
| Eudragit S100^{d} | 4.7 | 4.7 | 4.7 | 4.7 | 4.7 | 4.7 |
| Triethyl citrate | 2.3 | 2.3 | 2.3 | 2.3 | 2.3 | 2.3 |
| Talc | 2.3 | 2.3 | 2.3 | 2.3 | 2.3 | 2.3 |
| Purified Water | —^{b} | —^{b} | —^{b} | —^{b} | —^{b} | —^{b} |

Aesthetic Coat Suspension

| | | | | | | |
|---|---|---|---|---|---|---|
| Opadry II TBD ^{c, e} | — | 4 | — | 4 | — | 4 |
| Purified Water | — | —^{b} | — | —^{b} | — | —^{b} |

^{a}The actual quantity of linaclotide drug substance is adjusted based on the purity of the drug substance with a concomitant adjustment of microcrystalline cellulose
^{b}Purified water is removed during drug product processing
^{c} Represents 4% weight gain on previous step
^{d}Represents 9.5% total weight gain on previous step
^{e} Final Opadry may be either Blue 85F99031, Yellow 85F120017, or Orange 85F130136

### Example 9

### 300 μg or 600 μg Linaclotide Tablet Formulations

Delayed release 300 μg or 600 μg tablets can be prepared with the proportions of excipients as shown in Table 23 and Table 24. The tablets include a tablet core, a barrier coat, a functional coat, and an aesthetic coat.

TABLE 23
Tablet formulations 300 or 600 μg

| | % w/w | |
|---|---|---|
| Component | 300 μg | 600 μg |

Tablet Core

| | | |
|---|---|---|
| linaclotide^{a} | 0.066 | 0.132 |
| L-Histidine | 1.49 | 1.49 |
| Calcium Chloride Dihydrate | 0.71 | 0.71 |
| Polyvinyl Alcohol | 3.59 | 3.59 |
| MCC PH 200^{a} | 41.68 | 41.61 |
| Mannitol 100SD | 46.22 | 46.22 |
| Crosscarmellose sodium | 5.00 | 5.00 |
| Magnesium Stearate | 1.25 | 1.25 |
| Purified Water | —^{b} | —^{b} |
| Total (Tablet Core) | 100.0 | 100.0 |

Barrier Coat Suspension

| | | |
|---|---|---|
| Opadry II White 85F18422 | 3 | 3 |
| Purified Water | —^{b} | —^{b} |

Functional Coat Suspension

| | | |
|---|---|---|
| Eudragit S100^{d} | 2.9 | 2.9 |
| Eudragit L100^{d} | 0.5 | 0.5 |
| Triethyl citrate | 1.8 | 1.8 |
| Talc | 1.8 | 1.8 |
| Purified Water | —^{b} | —^{b} |

Aesthetic Coat Suspension

| | | |
|---|---|---|
| Opadry II TBD^{c, e} | 3 | 3 |
| Purified Water | —^{b} | —^{b} |

^{a}The actual quantity of linaclotide drug substance is adjusted based on the purity of the drug substance with a concomitant adjustment of microcrystalline cellulose
^{b}Purified water is removed during drug product processing
^{c}Represents 3% weight gain on previous step
^{d}Represents 7.1% total Eudragit weight gain on previous step
^{e}Final Opadry may be either Blue 85F99031, Yellow 85F120017, or Orange 85F130136

TABLE 24
Tablet formulation

| | % w/w | |
|---|---|---|
| Component | 300 μg | 600 μg |

Tablet Core

| | | |
|---|---|---|
| linaclotide^{a} | 0.066 | 0.132 |
| L-Histidine | 1.49 | 1.49 |
| Calcium Chloride Dihydrate | 0.71 | 0.71 |
| Polyvinyl Alcohol | 1.88 | 1.88 |
| MCC PH 200^{a} | 41.68 | 41.61 |
| Mannitol 100SD | 47.94 | 47.94 |
| Crosscarmellose sodium | 5.00 | 5.00 |
| Magnesium Stearate | 1.25 | 1.25 |
| Purified Water | —^{b} | —^{b} |
| Total (Tablet Core) | 100.0 | 100.0 |

Barrier Coat Suspension

| | | |
|---|---|---|
| Opadry II White 85F18422 | 3^{c} | 3^{c} |
| Purified Water | —^{b} | —^{b} |

Functional Coat Suspension

| | | |
|---|---|---|
| Eudragit S100^{d} | 3.5 | 3.5 |
| Triethyl citrate | 1.8 | 1.8 |
| Talc | 1.8 | 1.8 |
| Purified Water | —^{b} | —^{b} |

Aesthetic Coat Suspension

| | | |
|---|---|---|
| Opadry II TBD^{c, e} | 3 | 3 |
| Purified Water | —^{b} | —^{b} |

^{a}The actual quantity of linaclotide drug substance is adjusted based on the purity of the drug substance with a concomitant adjustment of microcrystalline cellulose
^{b}Purified water is removed during drug product processing
^{c}Represents 3% weight gain on previous step
^{d}Represents 7.1% total weight gain on previous step
^{e}Final Opadry will be either Blue 85F99031, Yellow 85F120017, or Orange 85F130136

### Example 10

### Administration of Linaclotide Targeting Visceral Pain Associated with IBS-d

Delayed release “DR2” linaclotide tablets were administered in a double-blind, placebo-controlled Phase 1 study randomized healthy adult volunteers at dosing levels of 300 μg, 1200 μg, and 3000 μg or placebo for 7 days. Safety and tolerability were assessed at each dose and prior to escalation to the highest dose. Gastrointestinal pharmacodynamics (“PD”) assessments included change from baseline (CFB) in bowel movement frequency (Spontaneous bowel movement [SBM]/week) and stool consistency (Bristol stool form scale [BSFS]). To confirm release of linaclotide, all of the subject's stools were inspected for intact DR2 tablets.

Trial Methodology:

The study was a Phase 1, single-center, randomized, double-blind, placebo-controlled, multiple dose study of linaclotide DR2 in healthy adult volunteers. The linaclotide DR2 tablets are designed to decrease the fluid secretion effects of linaclotide relative to immediate release (LINZESS®) capsules by targeting release of active drug near the ileocecal junction. Each cohort progressed through 3 study periods: (1) Screening Period, (2) Clinic Period, and (3) Follow-up Period. The study included up to 4 cohorts with 8 subjects per cohort. The 8 subjects in each cohort were randomized for treatments with linaclotide DR2 (6 subjects) or placebo (2 subjects) for 7 days. Cohorts 1 and 2 were enrolled concurrently and subsequent cohorts (Cohort 3 and Cohort 4) were enrolled sequentially. Dosing in Cohorts 3 and 4 proceeded following a safety review of prior dosed cohorts, as described below.

Subjects in each cohort were dosed approximately 30 minutes following the start of a standard breakfast on each dosing day. Study subjects received orally administered study drug (linaclotide DR2 or placebo) once daily for a total of 7 days during the Clinic Period, with the first dose administered on Day 1. Study drug was administered after an overnight fast of at least 10 hours and approximately 30 minutes following the start of a standard breakfast in the morning, with approximately 240 mL (8 ounces) of water. Food was not permitted for 4 hours after study drug administration and permitted concomitant medications were only be taken ≥2 hours after study drug administration.

Study drug was in the form of white, round, oral tablets. For the double-blind Clinic Period, subjects were supplied with identically appearing tablets containing linaclotide DR2 300 μg or placebo, as follows:
Cohort 1: 1 tablet linaclotide DR2 (300 μg) or placebo
Cohort 2: 4 tablets linaclotide DR2 (300 μg each; 1200 μg total) or placebo
Cohort 3: 10 tablets linaclotide DR2 (300 μg each; 3000 μg total) or placebo
Cohort 4 (optional): linaclotide DR2 (dose level determined based on safety review of previous cohorts) or placebo tablets

Linaclotide DR2 was provided as 300 μg oral tablets containing active pharmaceutical ingredient with the following excipients: microcrystalline cellulose, mannitol, croscarmellosesodium, polyvinyl alcohol, calcium chloride dihydrate, l-histidine, talc, polyethylene glycol, methacrylic acid copolymer type A and type B, triethyl citrate, titanium dioxide, and magnesium stearate. Matching placebo was provided as oral tablets containing the following excipients: microcrystalline cellulose, mannitol, croscarmellose sodium, polyvinyl alcohol, calcium chloride dihydrate, l-histidine, talc, polyethylene glycol, methacrylic acid copolymer type A and type B, triethyl citrate, titanium dioxide, and magnesium stearate.

Study Procedures

Safety and tolerability was evaluated through physical examination (including body height and weight), vital signs (blood pressure, pulse rate, oral temperature), and clinical laboratory evaluations (clinical chemistry, hematology, and urinalysis), and ECGs will be performed at designated timepoints during the Screening and Clinic Periods according to the Schedule of Evaluations). Subject- and investigator reported AEs will be recorded throughout the study.

PK assessments were conduct through blood determination of linaclotide and its metabolite in plasma, and feces for the determination of linaclotide and its metabolite in stool, were collected from all dosed subjects during the Clinic Period.

Bowel Movement Diary (BMD) treated subjects entered BM-related information into a paper diary on an event-driven basis (i.e. following each BM) during the Screening Period (beginning at Day −8) and throughout the Clinic Period. The information includeed the day and time of BMs and a self-report of stool consistency for each BM using the Bristol Stool Form Scale (BSFS; 1=Separate hard lumps like nuts [difficult to pass] to 7=Watery, no solid pieces [entirely liquid]).

Biomarkers from blood samples were collected during the Clinic Period for PK assessment and stored for up to 1 year for the assessment of PD biomarkers (eg, cyclic guanosine monophosphate [cGMP]) in plasma.

Trial Results:

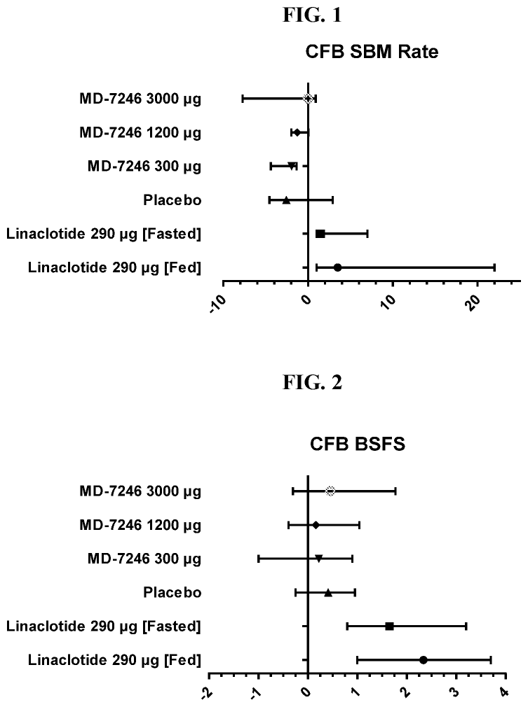

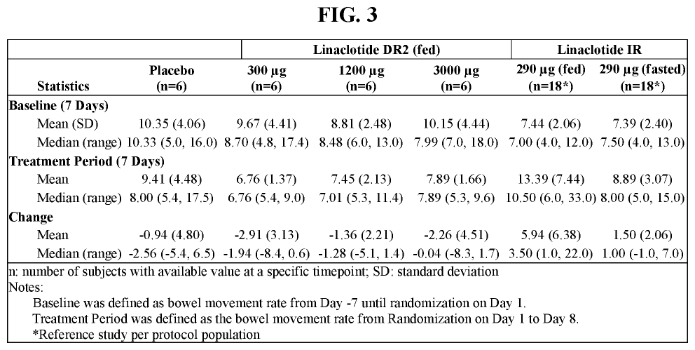

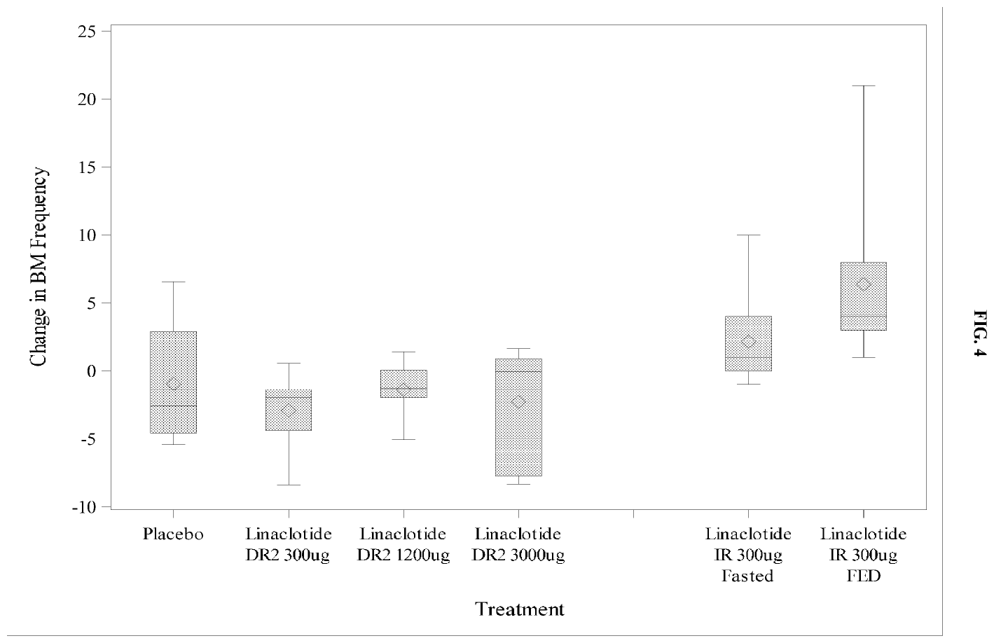

Placebo, 300 μg, 1200 μg, and 3000 μg DR2 tables were received by 6 subjects each. DR2 was well tolerated indicating a benign safety profile and there were no adverse events of diarrhea reported in any of the subjects who received either DR2 or placebo. Linaclotide was minimally absorbed with negligible systemic exposure. Neither linaclotide nor its active metabolite were detected in plasma following 7 days of oral administration and no intact tablets were discovered in the stool of any subject. Changes in SBM in BSFS were shown to be similar from baseline to end of treatment in placebo and all 3 DR2 dose groups (median CFB in SBM: −2.6, −1.9, −1.3, and 0.0; and median CFB BSFS: 0.4, 0.2, 0.2 and 0.4 in placebo, 300 μg, 1200 μg, and 3000 μg “DR2”, respectively). FIGS. 1-4 show these trial results.

For comparison the PD effects of immediate release formulation of linaclotide (290 μg) was evaluated in a previous food effects study in healthy volunteers (n=18) and showed SBM frequency and BSFS increased from baseline (median SBM CFB: 3.5 and 1.0; and median BSFS CFB: 2.34 and 1.65 in fed and fasted states, respectively). The trial demonstrated DR2 tablets, a delayed-release formulation of linaclotide was safe and well tolerated for 7 days at all 3 doses. DR2 demostrates distinct properties from the approved, immediate release linaclotide formulation and showed no effect on SBM frequency or stool consistency. In comparison, SBM frequency increased and stool consistency shifted to looser stools in healthy volunteers following linaclotide 290 μg for 7 days. Additionally, in contrast to the healthy volunteers who received of linaclotide 290 μg, there were no adverse events of diarrhea in any subject who received DR or placebo. DR2 demonstrated improvement on abdominal symptoms as shown in Table 25.

TABLE 25
Improvement of DR2 on Abdominal Symptoms

| | | Linaclotide DR2 | | |
|---|---|---|---|---|
| | Placebo | 300 ug | 1200 ug | 3000 ug |
| System Organ Class | (N = 6) | (N = 6) | (N = 6) | (N = 6) |
| Preferred Term | n (%) | n (%) | n (%) | n (%) |
| Subjects with at least one | 3 (50.0) | 0 | 0 | 2 (33.3) |
| Related TEAE | | | | |
| Gastrointestinal disorders | 3 (50.0) | 0 | 0 | 1 (16.7) |
| Abdominal distension | 1 (16.7) | 0 | 0 | 0 |
| Abdominal pain | 1 (16.7) | 0 | 0 | 0 |
| Dyspepsia | 1 (16.7) | 0 | 0 | 0 |
| Flatulence | 0 | 0 | 0 | 1 (16.7) |
| Nausea | 1 (16.7) | 0 | 0 | 0 |
| Nervous system disorders | 0 | 0 | 0 | 1 (16.7) |
| Headache | 0 | 0 | 0 | 1 (16.7) |

### Example 11

### Administration of Linaclotide for the Treatment of Abdominal Pain Associated Irritable Bowel Syndrome with Diarrhea (IBS-d)

Delayed release tablets will be administered to evaluate the safety and tolerability, treatment effect on abdominal pain, and dose response of DR2. DR2 will be administered orally to patients with diarrhea predominant irritable bowel syndrome (IBS-d). An objective of the study is to assess bowel function changes with DR2 in patients with IBS-d.

Trial Methodology:

This study is a multicenter, randomized, double-blind, placebo-controlled, parallel-group, dose-range-finding, 12-week study, consisting of 4 distinct periods, as illustrated in the figure below. The study will enroll patients who have IBS-d diagnosed using Rome IV criteria. Eligible patients will be randomized in equal proportions to 1 of 4 treatments: 300, 600, or 1200 μg of DR2, or matching placebo, administered once daily. DR2 is a delayed release tablet formulation of linaclotide designed to release linaclotide in the distal ileum near the ileocecal junction, to target guanylate cyclase C (GC-C) receptors in the colon and minimize secretory effects in the gastrointestinal (GI) tract.

Screening Period:

The Screening Period starts with the signing of the informed consent form and may last for up to 28 days. During this period, patient eligibility for entry into the Pretreatment Period will be determined. Loperamide, a protocol-permitted over-the-counter (OTC) medication for diarrhea, will be distributed to eligible patients beginning at the Screening Visit. The end of the Screening Period coincides with the start of the Pretreatment Period, if the patient meets the entry criteria assessed at the Screening Visit and does not require a washout of prohibited medicines.

Pre-Treatment Period:

The Pretreatment Period is defined as the 14 to 21 days immediately before a randomization visit. During this period, patients will provide the following information in a handheld electronic diary (eDiary):

Daily Assessments:
Daily Abdominal Symptom Assessments in a daily evening report
Bowel Movement (BM)-related Assessments on an event-driven basis (meaning these are assessments made for each event at the time the event occurs or during the daily evening report for any events not previously entered for that day)

Weekly Assessments:
Weekly Patient Assessment of Degree of Relief of IBS Symptoms
Weekly Patient Assessment of Adequate Relief of IBS Pain
Weekly Patient Assessment of BM-related Symptom Severity (patient global impression of severity [PGI-S])
Weekly Patient Assessment of BM-related Symptom Change (patient global impression of change [PGI-C])

Use of Loperamide for Diarrhea on an event-driven basis:

Patients who satisfy all entry criteria will enter the Treatment Period.

Treatment Period:

The Treatment Period begins with treatment assignment and lasts for 12 weeks. Patients will be randomized in equal proportions to 1 of 4 treatments: 300, 600, or 1200 μg of DR2, or matching placebo. Patients will take their initial dose of study drug at the study center during the Randomization Visit. On all other days, study drug will be taken once daily at approximately the same time of day without regard to food (patients will be instructed to choose a time that is convenient for them and continue daily dosing at that time throughout the Treatment Period). Patients will continue to use the handheld eDiary to provide their:

Daily Assessments:
Daily Abdominal Symptom Assessments in a daily evening report
BM-related Assessments on an event-driven basis (meaning these are assessments made for each event at the time the event occurs [or during the daily evening report for any events not previously entered for that day]) Weekly Assessments:

Weekly Patient Assessment of Degree of Relief of IBS Symptoms
Weekly Patient Assessment of Adequate Relief of IBS Pain
Weekly Patient Assessment of Treatment Satisfaction
Weekly Patient Assessment of BM-related Symptom Severity (PGI-S)
Weekly Patient Assessment of BM-related Symptom Change (PGI-C)
Use of Loperamide for Diarrhea on an event-driven basis.

Health-related quality-of-life and patient-outcome assessments will be performed at the randomization visit and at study visits throughout the Treatment Period. Patients will complete a Week 2 Phone Call, and Week 4, Week 8, and Week 12/End of Treatment (EOT) Visits during the Treatment Period.

Post-Treatment Period:

The Post-treatment period starts on the day following the last day of dosing (Week 12/EOT Visit) and finishes 2 weeks later at the end of study (EOS) Follow-up Phone Call. During the call, patients will be asked to report any AEs and medicines taken since the Week 12/EOT Visit and detail any other symptoms or comments they may have (at the discretion of the investigator, patients may be requested to return to the study center for their EOS Follow-up).

Study Procedures

During the Pretreatment and Treatment Periods, patients will enter information into the eDiary. Certain information will be entered by the patient on an event-driven basis, in a daily evening report, and on a weekly basis, as specified below. Daily Assessments: The following information will be entered by the patient into the eDiary each day:
Diary of IBS Symptoms—Diarrhea
   Daily Abdominal Symptom Assessments (entered by the patient in a daily evening report):
      Rating of abdominal bloating at its worst during the previous 24 hours on an 11-point numerical rating scale (NRS)
      Rating of abdominal discomfort at its worst during the previous 24 hours on an 11-point NRS
      Rating of abdominal pain at its worst during the previous 24 hours on an 11-point NRS
      Rating of abdominal cramping at its worst during the previous 24 hours on an 11-point NRS
   Daily BM-related Assessments (entered by the patient for each BM on an event-driven basis):
      BM date and time
      Stool consistency on a 5-point ordinal scale
      Stool consistency on the 7-point Bristol Stool Form Scale (BSFS)
      BM urgency on a binary (Yes/No) scale
Use of Loperamide for Diarrhea on an event-driven basis

Weekly Assessments:

The following information will be entered by the patient into the eDiary once per week at the same time as the daily evening report:
Patient Assessment of Degree of Relief of IBS Symptoms on a 7-point balanced scale
Patient Assessment of Adequate Relief of IBS Pain on a binary scale (Yes/No)
Patient Assessment of Treatment Satisfaction on a 5-point ordinal scale (assessed at each week of the Treatment Period after the Randomization Visit)
Patient Assessment of BM-related Symptom Severity on a 5-point ordinal scale
Patient Assessment of BM-related Symptom Change on a 7-point ordinal scale

### Other Embodiments

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

We claim:

1. A method of treating visceral or abdominal pain in a non-constipated subject, comprising orally administering to a patient in need thereof, a pharmaceutical tablet composition comprising a therapeutically effective amount of linaclotide.

2. A method of treating irritable bowel syndrome with diarrhea (IBS-d) comprising orally administering to a patient in need thereof, a pharmaceutical tablet composition comprising a therapeutically effective amount of linaclotide.

3. A method of reducing intestinal fluid secretion-promoting effects of linaclotide, comprising orally administering to a subject a pharmaceutical tablet composition comprising linaclotide and the tablet further comprises an enteric coating comprising a pH-sensitive polymer that releases linaclotide in a lower GI of the subject.

4. A method of treating abdominal pain comprising orally administering to a patient in need thereof, a pharmaceutical tablet composition comprising a therapeutically effective amount of linaclotide and wherein the tablet further comprises an enteric coating comprising a pH-sensitive polymer that releases linaclotide in a lower GI of the subject.

5. The method of any of the preceding claims, wherein the pharmaceutical tablet composition comprises a therapeutically effective amount of linaclotide to reduce, prevent or relieve pain or diarrhea in the subject.

6. The method of any of the preceding claims, wherein the pharmaceutical tablet composition comprises a therapeutically effective amount of linaclotide to reduce, prevent or relieve pain in the subject, but does not affect bowel habit.

7. The method of claim 6, wherein the bowel habit is selected from CSBM rate, SBM rate, or stool consistency.

8. The method of any of the preceding claims, wherein the subject is diagnosed with irritable bowel syndrome with diarrhea (IBS-d).

9. The method of any of the preceding claims, wherein the linaclotide is present in the delayed-release pharmaceutical tablet composition in an amount between 30 μg to 5,000 μg.

10. The method of claim 9, wherein the linaclotide is present in an amount of about 300 μg, about 600 μg, about 1200 μg, or about 3000 μg.

11. The method of any of the preceding claims, wherein the pharmaceutical tablet composition further comprises between 0%-2% per weight of an amino acid selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, or any mixture thereof.

12. The method any of the preceding claims, wherein the pharmaceutical tablet composition further comprises between 0%-2% or between 0.5%-1.5% per weight of histidine.

13. The method of any of the preceding claims, wherein the pharmaceutical tablet composition further comprises between 0%-3% per weight of a cation salt selected from the group consisting of calcium, potassium, magnesium, zinc, aluminum, manganese, chromium, cobalt, nickel, barium, and sodium, or any combination or mixture thereof.

14. The method of any of the preceding claims, wherein the pharmaceutical tablet composition further comprises between 0%-3% per weight of a calcium salt.

15. The method of any of the preceding claims, wherein the pharmaceutical tablet composition further comprises between 0%-2% or between 0.2%-0.8% per weight of calcium chloride dehydrate.

16. The method of any of the preceding claims, wherein the pharmaceutical tablet composition further comprises between 0%-5%, between 1%-5%, or between 1%-1.88% per weight of polyvinyl alcohol (PVA).

17. The method of any of claims 3-16, wherein the pH-sensitive polymer has a dissolution pH of at least 6.0, at least 6.5, or at least 7.0

18. The method of any of claims 3-17, wherein the pH-sensitive polymer comprises a methyl acrylate-methacrylic acid copolymer (e.g., Eudragit®).

19. The method of any of claims 3-18, wherein the pH-sensitive polymer comprises Eudragit S100.

20. The method of any of claims 3-19, wherein the pH-sensitive polymer comprises Eudragit L100.

21. The method of any of claims 3-18, wherein the pH-sensitive polymer consists essentially of Eudragit S100.

22. The method of any of claims 3-18, wherein the pH-sensitive polymer comprises a mixture of Eudragit S100 and Eudragit L100.

23. The method of claim 22, wherein the pH-sensitive polymer comprises a mixture of Eudragit S100 and Eudragit L100 at a ratio of between 1:1 and 6:1 (S100:L100), at a ratio of between 4.5:1 and 5.5:1 (S100:L100), or at a ratio of 4.875:1 (S100:L100) by weight.

24. The method of any of the preceding claims, wherein the pharmaceutical tablet composition comprises an enteric coated tablet.

25. The method of any of the preceding claims, wherein the delayed release pharmaceutical tablet composition comprises:
Ca²⁺;
histidine; and
polyvinyl alcohol (PVA).

26. The method of any of the preceding claims, further comprising a protective polymer film or subcoating.

27. The method of claim 26, wherein the subcoating comprises Opadry II®.

28. The method of any of the preceding claims, wherein the pharmaceutical tablet composition is administered once daily.

29. The method of any of the preceding claims, wherein the pharmaceutical tablet composition is administered once daily in the morning.

30. The method of any of the preceding claims, wherein the pharmaceutical tablet composition is administered once daily in the morning at least 30 minutes after breakfast.

31. The method of any of the preceding claims, wherein the pharmaceutical tablet composition is administered after the patient has fasted for at least 12 hours.

32. The method of any of the preceding claims, wherein the pharmaceutical tablet composition is administered for at least 6 weeks, at least 7 weeks, or at least 12 weeks.

33. The method of any of the preceding claims, wherein the administering decreases abdominal pain in the patient.

34. The method of any of the preceding claims, wherein the administering decreases abdominal discomfort in the patient.

35. The method of any of the preceding claims, wherein the administering decreases abdominal bloating in the patient.

36. The method of any of the preceding claims, wherein the administering decreases abdominal cramping in the patient.

37. The method of any of the preceding claims, wherein the administering improves two or more of the following: abdominal pain, abdominal discomfort, abdominal bloating, cramping, abdominal symptom score, IBS symptom severity, treatment satisfaction, and assessment of adequate relief.

38. The method of any of the preceding claims, wherein the administering decreases the abdominal pain in the patient from baseline by at least 30% during at least 6 out of 12 weeks.

39. The method of any of the preceding claims, wherein the administering does not change one or more of the following: CSBM frequency rate, SBM frequency rate, or stool consistency.

40. A method of treating or relieving pain comprising administering to a patient in need thereof a therapeutically effective amount of a delayed-release pharmaceutical tablet composition of any of the preceding claims.

41. The method of claim 40, wherein the pain is selected from visceral pain; diverticulitis pain; pelvic pain; abdominal pain; or pain associated with gastrointestinal disorders, venereal diseases, bladder pain syndrome, or interstitial cystitis.

42. The method of claim 40, wherein the pain is selected from general abdominal pain, diverticular disease, pain associated with irritable bowel syndrome (IBS), chronic or acute radiation proctopathy (also referred to as radiation proctitis), rectal pain, chronic proctalgia, proctalgia fugax, anal pain, chronic anal fissure, post-operative anal pain, overactive bladder syndrome, stress incontinence, interstitial cystitis, bladder pain syndrome, pain associated with cancer, pain associated with gastrointestinal tract neoplasms, general pelvic pain, endometriosis, orchialgia, chronic prostatitis, prostatodynia, vulvodynia, urethral syndrome, penile pain, perianal pain, and pain associated with ulcerative colitis, ulcerative proctitis, or Crohn's disease.

43. The method of claim 42, wherein the pain is associated with irritable bowel syndrome.

44. The method of claim 43, wherein the irritable bowel syndrome is irritable bowel syndrome with diarrhea (IBS-d).

45. The method of any one of claims 1-43, wherein the pharmaceutical tablet composition releases linaclotide in the lower GI.

46. The method of any one of claims 1-43, wherein the pharmaceutical tablet composition releases linaclotide in the ileum, terminal ileum, or colon.

47. The method of any one of claims 1-43, wherein the pharmaceutical tablet composition releases linaclotide in the distal ileum near the ileocecal junction.
